SEQUENCE LISTING (16 sequences)

SEQ ID NO: 1 - DNA, 9456 bp, Hepatitis C virus
  CDS (340)..(9405)
  ref <308>: DATABASE ACCESSION NUMBER: Genbank / D17763
  ref <309>: DATABASE ENTRY DATE: 1993-09-27
  ref <313>: RELEVANT RESIDUES: (1)..(9456)
acctgcctcttacgaggcgacactccaccatggatcactcccctgtgaggaacttctgtcttcacgcggaaagcgcctagccatggcgttagtacgagtgtcgtgcagcctccaggaccccccctcccgggagagccatagtggtctgcggaaccggtgagtacaccggaatcgctggggtgaccgggtcctttcttggagcaacccgctcaatacccagaaatttgggcgtgcccccgcgagatcactagccgagtagtgttgggtcgcgaaaggccttgtggtactgcctgatagggtgcttgcgagtgccccgggaggtctcgtagaccgtgcaacatgagcacacttcctaaacctcaaagaaaaaccaaaagaaacaccatccgtcgcccacaggacgtcaagttcccgggtggcggacagatcgttggtggagtatacgtgttgccgcgcaggggcccacgattgggtgtgcgcgcgacgcgtaaaacttctgaacggtcacagcctcgcggacgacgacagcctatccccaaggcgcgtcggagcgaaggccggtcctgggctcagcccgggtacccttggcccctctatggtaacgagggctgcgggtgggcagggtggctcctgtccccacgcggctcccgtccatcctggggcccaaatgacccccggcggaggtcccgcaatttgggtaaagtcatcgataccctaacgtgcggattcgccgacctcatggggtacatcccgctcgtcggcgctcctgtaggaggcgtcgcaagagccctcgcgcatggcgtgagggcccttgaagacgggataaatttcgcaacagggaacttgcccggttgctccttttctatcttccttcttgctctgttctcttgcttaattcatccagcagccagtctagagtggcggaatacgtctggcctctacgtccttaccaacgactgttccaatagcagtattgtgtatgaggccgatgatgtcattctgcacacacccggctgtgtaccttgtgtccaggacggcaatacatctacgtgctggaccccagtgacacctacagtggcagtcaggtacgtcggagcaactactgcttcgatacgcagtcatgtggacctattagtaggcgcggccacgatgtgctctgcgctctacgtgggtgatatgtgtggggctgtctttctcgtgggacaagccttcacgttcagacctcgacgccatcaaacggtccagacctgtaactgctcgctgtacccaggccatctttcaggacatcgaatggcttgggatatgatgatgaattggtcccccgctgtgggtatggtggtggcgcatgtcctgcgtttaccccagaccttgttcgacataatggccggggcccattggggcatcttggcgggcctggcctattactccatgcagggcaactgggccaaggtcgcaatcatcatggttatgttctcaggggtcgatgcccacacatataccaccggtggcactgcatctcgtcatacccaagcgtttgctggtctttttgacataggcccccaacagaaactgcagctggtcaacaccaatggctcgtggcacatcaacagtactgccctaaattgcaatgagtccataaacaccgggtttatagctgggttgttttattaccataagttcaactctactggatgtcctcaaaggctcagcagctgcaagcccatcactttcttcaggcagggatggggccccttaacagatgctaacatcaccggtccttctgatgacagaccatactgctggcactacgcacctagaccttgtgacattgtcccggcatcaagtgtctgcggccctgtgtactgcttcacaccatcgccagtggtcgtaggcactactgatgccaggggcgtgccaacctacacctggggtgagaatgagaaagatgtgttcctgctgaagtcccagcggcctcccagtggtcggtggtttgggtgctcgtggatgaactccacggggtttctcaagacgtgcggagctcccccctgtaacatctatgggggcgaggggaatccccacaatgaatcagatcttttctgccccactgactgcttcaggaaacatcccgagaccacgtacagccggtgtggtgcagggccctggttgacacctcgttgcatggttgactacccataccggctttggcattacccatgtacagtcgatttcagattgttcaaggtgaggatgtttgtgggtgggtttgaacatcgatttaccgccgcttgcaactggaccaggggggagcgctgcgatatcgaggatcgtgaccgcagtgagcaacatccgctgctgcattcaacaactgagcttgctatactgccttgctctttcacgcccatgcctgcgctgtcaacaggtctgatacacctccaccaaaacatcgtggatgtccaatacctttatggcgttggatctggcatggtgggatgggcgctgaaatgggagttcgtcatcctcgttttcctccttctggcggacgcacgcgtgtgcgttgccctttggctgatgctgatgatatcacagacagaagcagccttggagaacctggtcacgctgaacgccgtcgctgctgctgggacacatggtatcggctggtacctggtagctttttgcgcggcgtggtacgtgcggggtaaactcgtcccgctggtgacctacagcctgacgggtctttggtccctagcattgctcgtcctcttgctcccccaacgtgcgtatgcttggtcgggtgaagacagcgccactcttggcgctggggtcttggtcctcttcggcttctttaccttgtcaccctggtataagcattggatcggccgcctcatgtggtggaaccagtacaccatatgcagatgcgagtccgcccttcacgtgtgggttccccccttactcgcacgcgggagtagggatggtgtcatcctgctaacaagcctgctttatccatccttaatttttgacatcactaagctgctgatggcagtattgggcccattatacttaatacaggctaccattactaccaccccctactttgtgcgtgcgcatgtactggtccgcctttgcatgctcgtgcgctccgtgataggggggaaatacttccagatgatcatactgagcattggcagatggttcaacacctacctatacgaccacctagcgccaatgcaacactgggccgctgctggtctcaaagacctagcagtggccactgaacctgtaatatttagtcccatggaaatcaaggtcatcacctggggtgcggatacagcggcttgcggagatattctttgcgggctgcccgtctctgcacgattaggccgtgaggtgttgttgggacctgctgatgactatcgggagatgggctggcgtctgttggccccgattacagcatacgcccagcaaactaggggccttcttgggactattgtgactagcttgactggcagagacaagaacgtggtgaccggtgaagtgcaggtgctttctacggctacccagaccttcctaggtacaacagtagggggggttatatggactgtttatcatggagcaggttcgagaacgctcgcgggcgccaaacatcccgcgctccaaatgtacacaaatgtagatcaggacctcgttgggtggccagcccctccaggggccaagtctcttgaaccgtgcgcctgcgggtcttcagacttatacttggttacccgcgatgccgatgtcatccctgctcggcgcagaggggactccacagcgagcttgctcagtcctaggcctctcgcctgtctcaagggttcctctggaggtcccgttatgtgcccttcggggcatgttgcggggatctttagggctgctgtgtgcaccagaggtgtagcaaaatccctacagttcataccagtggaaacccttagcacgcaggctaggtctccatctttctctgacaattcaactcctcctgctgttccacagagctatcaagtaggatacctccatgccccgaccggcagcggtaagagcacaaaggtcccggccgcttatgtagcacaaggatataatgtcctcgtactaaatccatcggtggcggccacattaggcttcggctccttcatgtcgcgtgcctatgggatcgaccccaacatccgcactgggaaccgcaccgttacaactggtgctaaactgacctattccacctacggtaagtttctcgcggacgggggttgctccgggggggcatatgatgtgattatctgtgatgaatgtcatgcccaagacgctactagcatattgggtataggcacggtcttagatcaggctgagacggctggggtgaggctgacggttttagcgacagcaactcccccaggcagcatcactgtgccgcattctaacatcgaagaagtggccctgggctctgaaggtgagatccctttctacggtaaggctataccgatagccctgctcaagggggggaggcaccttatcttttgccattccaagaaaaaatgtgatgagatagcgtccaaactaagaggcatggggctcaacgctgtagcatactataggggtctcgatgtgtccgtcataccaacaacaggagacgtcgtagtttgcgctactgacgccctcatgactggattcaccggggacttcgattctgtcatagattgcaacgtggctgttgaacagtacgttgacttcagcctggaccccaccttttccattgagacccgcactgctccccaagatgcggtttcccgcagccaacgtcgcggccgaacgggtcgaggtagactcggtacgtaccgatatgtcgcctccggtgaaagaccgtctggaatgtttgactcggtcgtcctctgtgagtgctatgacgcgggctgctcatggtacgatctgcagcccgctgagaccacagttagactgagagcttacttgtccacacccgggttacccgtctgccaagaccatttagacttttgggagagcgtcttcactggactaactcacatagatgcccactttctgtcacagactaagcagcagggacttaacttctcgtacctaactgcctaccaagccactgtgtgcgcccgcgcgcaggctcctcccccaagttgggacgagatgtggaagtgtctcgtgcggcttaagccaacactacatggacctacgccccttctatatcggctggggcctgtccaaaatgaaacctgcttgacacaccccatcacaaaatacctcatggcatgcatgtcagccgatctggaagtaaccaccagcacctgggtgttgctcggaggggtcctcgcagccctagcggcctactgcttgtcagtcggctgcgttgtgattgtgggtcatattgagctggagggcaagccagcactcgttccagacaaagaggtgttgtatcaacaatacgatgagatggaggagtgctcacaagctgccccatatatcgaacaagctcaggtaatagcccaccagttcaaggaaaaaatccttggattgctgcagcgagccacccagcaacaagctgtcattgagcccatagtaactaccaactggcaaaagcttgaggccttctggcacaagcatatgtggaattttgtgagtgggatccaatacctagcaggcctctccactttgcctggcaaccctgctgtggcgtctcttatggcgttcactgcttcagtcaccagtcccctgacgaccaaccaaactatgttttttaacatactcggggggtgggttgccacccatttggcagggccccagagctcctccgcgttcgtggtaagcggcttggcaggcgctgccatagggggtataggcctgggcagggtcttgcttgacatcctggcaggatacggagctggtgtctcaggcgccttggtagcttttaagatcatgggaggagaatgccccactgctgaggacatggtcaacctgttgcccgccatactatctccgggtgctctcgtcgtcggtgtgatatgcgcagccatactgcgccgacacgtaggacctggggagggagcggtacagtggatgaacaggctcatcgcgttcgcatcccggggcaaccacgtctcaccgacgcactatgttcccgagagcgatgctgcggcaagggtcaccgcattgctgagttctctaactgtcacaagtctgctccggcggttacaccagtggatcaatgaagactacccaagcccttgtagcgacgattggctacgtaccatctgggactgggtttgctcggtgttggccgacttcaaggcatggctctctgctaagattatgccagcgctccctgggctgcccttcatttcctgtcaaaagggatacaagggcgtgtggcggggggacggtgtgatgtcaacacgctgtccttgcggggcagcaataactggccatgtgaagaacgggtccatgcggcttgcagggccgcgtacatgtgctaacatgtggcacggtactttccccatcaatgagtacaccaccggacccagcacaccttgcccatcacccaactacactcgcgcactatggcgcgtggctgccaacagctacgttgaggtgcgtcgggtgggggacttccattacatcacgggggccacagaagatgagctcaagtgtccgtgccaagtgccggctgctgagttctttactgaagtggatggagtgagacttcaccgctacgcccctccatgtaagcccctgttgagagatgatatcactttcatggtagggttgcattcctacacgataggatctcaactcccctgtgagccagaaccggatgtctctgtgctgacctcgatgttgagagacccttcccatatcaccgccgagacggcagcgcgccgccttgcacgcgggtcccctccatcagaggcaagctcatccgccagccaactatcagctccgtcgttgaaggccacttgccagacgcataggcctcatccagacgctgagctagtggacgccaacttgttatggcggcaagagatgggcagcaacattacacgggtggagtctgagacaaaggttgtggttcttgattcgttcgagcctctgagagccgaaactgatgacgtcgagccctcggtggctgcagagtgtttcaagaaacctcccaagtatcctccagcccttcctatctgggctagaccggactacaatcctccactgttggaccgctggaaagcaccggattatgtaccaccaactgtccatggatgtgccttaccaccacggggcgctccaccggtgcctccccctcggaggaaaaggacaattcagctggacggctctaatgtgtccgcggcgctagccgcgctagctgaaaaatcattcccgtcctcgaaaccacaggaagagaatagctcatcctccggggtcgacacacagtccagcactacttccaaggtgcccccttctccgggaggggagtccgactcagagtcatgctcgtctatgcctcctctcgagggagagccgggcgacccggacttgagttgcgactcttggtccaccgttagtgacagcgaggagcagagcgtggtctgctgctctatgtcgtattcttggaccggcgccctgataacaccatgtagtgctgaggaagagaaactgcccatcagcccactcagcaactccctgttgagacatcataacctagtctattcaacgtcgtctagaagcgcttctcagcgtcagaagaaggttaccttcgatagactgcaggtgctcgacgaccattacaagactgcattaaaggaggtaaaggagcgagcgtctagggtaaaggctcgcatgctcaccatcgaggaagcgtgcgcgctcgtccctcctcactctgcccgatcgaagttcgggtatagtgcgaaggacgttcgctccttgtccagcagggccattaaccagatccgctccgtctgggaggacttgctggaagacaccacaactccaattccaaccaccatcatggcgaagaacgaggtgttttgcgtggaccccgctaaagggggccgcaagcccgctcgcctcattgtgtaccctgacctgggggtgcgtgtctgtgagaaacgcgccctgtatgacgtgatacagaagttgtcaattgagacgatgggtcctgcctatggattccaatactcgcctcaacagcgggtcgaacgtctgctgaagatgtggacctcaaagaaaacccccttggggttctcatatgacacccgctgctttgactcaactgtcactgaacaggacatcagggtggaagaggagatataccaatgctgtaaccttgaaccggaggccaggaaagtgatctcctccctcacggagcggctttactgcggaggccctatgttcaacagcaagggggcccagtgtggttatcgccgttgccgtgccagtggagttctgcctaccagcttcggcaacacaatcacttgttacatcaaggccacagcggctgcaaaggccgcaaacctccggaacccggactttcttgtctgcggagatgatttggtcgtggtggctgagagtgatggcgtcgatgaggatagagcagccctgagagccttcacggaggctatgaccaggtattctgctccacccggagatgctccacaggccacttacgaccttgagcttatcacatcttgctcctccaacgtctccgtggcacgggacgacaaggggaggaggtactattacctcacccgtgatgccactactcccctagcccgtgcggcttgggaaacagctcgtcacactccagttaactcctggttaggcaacatcatcatgtacgcgcctaccatctgggtgcgcatggtgatgatgacacactttttctccatactccaatcccaggagatacttgatcgcccccttgactttgaaatgtacggggccacttattctgtcactccgctggatttaccagcaatcattgaaagactccatggtctaagcgcgttcacgctccacagttactctccagtagagctcaatagggtcgcggggacactcaggaagcttgggtgcccccccctacgggcttggagacatcgggcacgagcagtgcgcgccaagcttatcgcccagggaggaaaggctaaaatatgcggcctttatctctttaattgggcggtacgcaccaagaccaacctcactccattgccagccgctggccagttggatttgtccagctggtttacggttggtgtcggcgggaacgacatttatcacagcgtgtcgcgtgcccgaacccgccatttgctgctttgcctactcctactaacagtaggggtaggcatctttctcttgccagctcgatgagctggtaagataacactccatttcttttttgtttttttttttttttttttt

SEQ ID NO: 2 - protein, 3021 aa, Hepatitis C virus
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCVPCVQDGNTSTCWTPVTPTVAVRYVGATTASIRSHVDLLVGAATMCSALYVGDMCGAVFLVGQAFTFRPRRHQTVQTCNCSLYPGHLSGHRMAWDMMMNWSPAVGMVVAHVLRLPQTLFDIMAGAHWGILAGLAYYSMQGNWAKVAIIMVMFSGVDAHTYTTGGTASRHTQAFAGLFDIGPQQKLQLVNTNGSWHINSTALNCNESINTGFIAGLFYYHKFNSTGCPQRLSSCKPITFFRQGWGPLTDANITGPSDDRPYCWHYAPRPCDIVPASSVCGPVYCFTPSPVVVGTTDARGVPTYTWGENEKDVFLLKSQRPPSGRWFGCSWMNSTGFLKTCGAPPCNIYGGEGNPHNESDLFCPTDCFRKHPETTYSRCGAGPWLTPRCMVDYPYRLWHYPCTVDFRLFKVRMFVGGFEHRFTAACNWTRGERCDIEDRDRSEQHPLLHSTTELAILPCSFTPMPALSTGLIHLHQNIVDVQYLYGVGSGMVGWALKWEFVILVFLLLADARVCVALWLMLMISQTEAALENLVTLNAVAAAGTHGIGWYLVAFCAAWYVRGKLVPLVTYSLTGLWSLALLVLLLPQRAYAWSGEDSATLGAGVLVLFGFFTLSPWYKHWIGRLMWWNQYTICRCESALHVWVPPLLARGSRDGVILLTSLLYPSLIFDITKLLMAVLGPLYLIQATITTTPYFVRAHVLVRLCMLVRSVIGGKYFQMIILSIGRWFNTYLYDHLAPMQHWAAAGLKDLAVATEPVIFSPMEIKVITWGADTAACGDILCGLPVSARLGREVLLGPADDYREMGWRLLAPITAYAQQTRGLLGTIVTSLTGRDKNVVTGEVQVLSTATQTFLGTTVGGVIWTVYHGAGSRTLAGAKHPALQMYTNVDQDLVGWPAPPGAKSLEPCACGSSDLYLVTRDADVIPARRRGDSTASLLSPRPLACLKGSSGGPVMCPSGHVAGIFRAAVCTRGVAKSLQFIPVETLSTQARSPSFSDNSTPPAVPQSYQVGYLHAPTGSGKSTKVPAAYVAQGYNVLVLNPSVAATLGFGSFMSRAYGIDPNIRTGNRTVTTGAKLTYSTYGKFLADGGCSGGAYDVIICDECHAQDATSILGIGTVLDQAETAGVRLTVLATATPPGSITVPHSNIEEVALGSEGEIPFYGKAIPIALLKGGRHLIFCHSKKKCDEIASKLRGMGLNAVAYYRGLDVSVIPTTGDVVVCATDALMTGFTGDFDSVIDCNVAVEQYVDFSLDPTFSIETRTAPQDAVSRSQRRGRTGRGRLGTYRYVASGERPSGMFDSVVLCECYDAGCSWYDLQPAETTVRLRAYLSTPGLPVCQDHLDFWESVFTGLTHIDAHFLSQTKQQGLNFSYLTAYQATVCARAQAPPPSWDEMWKCLVRLKPTLHGPTPLLYRLGPVQNETCLTHPITKYLMACMSADLEVTTSTWVLLGGVLAALAAYCLSVGCVVIVGHIELEGKPALVPDKEVLYQQYDEMEECSQAAPYIEQAQVIAHQFKEKILGLLQRATQQQAVIEPIVTTNWQKLEAFWHKHMWNFVSGIQYLAGLSTLPGNPAVASLMAFTASVTSPLTTNQTMFFNILGGWVATHLAGPQSSSAFVVSGLAGAAIGGIGLGRVLLDILAGYGAGVSGALVAFKIMGGECPTAEDMVNLLPAILSPGALVVGVICAAILRRHVGPGEGAVQWMNRLIAFASRGNHVSPTHYVPESDAAARVTALLSSLTVTSLLRRLHQWINEDYPSPCSDDWLRTIWDWVCSVLADFKAWLSAKIMPALPGLPFISCQKGYKGVWRGDGVMSTRCPCGAAITGHVKNGSMRLAGPRTCANMWHGTFPINEYTTGPSTPCPSPNYTRALWRVAANSYVEVRRVGDFHYITGATEDELKCPCQVPAAEFFTEVDGVRLHRYAPPCKPLLRDDITFMVGLHSYTIGSQLPCEPEPDVSVLTSMLRDPSHITAETAARRLARGSPPSEASSSASQLSAPSLKATCQTHRPHPDAELVDANLLWRQEMGSNITRVESETKVVVLDSFEPLRAETDDVEPSVAAECFKKPPKYPPALPIWARPDYNPPLLDRWKAPDYVPPTVHGCALPPRGAPPVPPPRRKRTIQLDGSNVSAALAALAEKSFPSSKPQEENSSSSGVDTQSSTTSKVPPSPGGESDSESCSSMPPLEGEPGDPDLSCDSWSTVSDSEEQSVVCCSMSYSWTGALITPCSAEEEKLPISPLSNSLLRHHNLVYSTSSRSASQRQKKVTFDRLQVLDDHYKTALKEVKERASRVKARMLTIEEACALVPPHSARSKFGYSAKDVRSLSSRAINQIRSVWEDLLEDTTTPIPTTIMAKNEVFCVDPAKGGRKPARLIVYPDLGVRVCEKRALYDVIQKLSIETMGPAYGFQYSPQQRVERLLKMWTSKKTPLGFSYDTRCFDSTVTEQDIRVEEEIYQCCNLEPEARKVISSLTERLYCGGPMFNSKGAQCGYRRCRASGVLPTSFGNTITCYIKATAAAKAANLRNPDFLVCGDDLVVVAESDGVDEDRAALRAFTEAMTRYSAPPGDAPQATYDLELITSCSSNVSVARDDKGRRYYYLTRDATTPLARAAWETARHTPVNSWLGNIIMYAPTIWVRMVMMTHFFSILQSQEILDRPLDFEMYGATYSVTPLDLPAIIERLHGLSAFTLHSYSPVELNRVAGTLRKLGCPPLRAWRHRARAVRAKLIAQGGKAKICGLYLFNWAVRTKTNLTPLPAAGQLDLSSWFTVGVGGNDIYHSVSRARTRHLLLCLLLLTVGVGIFLLPAR

SEQ ID NO: 3 - DNA, 34 bp, Artificial sequence
  Artificially synthesized oligonucleotide primer
atgcgaattcgccaccatgagcacacttcctaaa

SEQ ID NO: 4 - DNA, 31 bp, Artificial sequence
  Artificially synthesized oligonucleotide primer
agtctctagatcatcaacttgctgctggatg

SEQ ID NO: 5 - DNA, 43 bp, Artificial sequence
  Artificially synthesized oligonucleotide primer
agtctctagatcatcaacttgctgctggatgaattaagcaaga

SEQ ID NO: 6 - DNA, 43 bp, Artificial sequence
  Artificially synthesized oligonucleotide primer
agtctctagatcatcaacttgctgctggatgaactaagcaaga

SEQ ID NO: 7 - DNA, 58 bp, Artificial sequence
  Artificially synthesized oligonucleotide primer
agtctctagatcatcaacttgctgctggatgaattaagcaagagaacagagctagaag

SEQ ID NO: 8 - DNA, 20 bp, Artificial sequence
  Artificial PCR primer
cgcaaatgggcggtaggcgt

SEQ ID NO: 9 - DNA, 20 bp, Artificial sequence
  Artificial PCR primer
tctctgtaggtagtttgtcc

SEQ ID NO: 10 - DNA, 9616 bp, Hepatitis C virus
  CDS (342)..(9389)
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccacaggacgtcaagttcccgggcggtggtcagatcgttggtggagtttacctgttgccgcgcaggggccccaggttgggtgtgcgcgcgatcaggaagacttccgagcggtcgcaaccccgtggaaggcgacagcctatccccaaggctcgccggcccgagggcagggcctgggctcagcccgggtatccttggcccctctatggcaatgagggcatggggtgggcaggatggctcctgtcaccccgcggctcccggcctagttggggccccacggacccccggcgtaggtcgcgtaatttgggtaaggtcatcgataccctcacatgcggcctcgccgacctcatggggtacattccgctcgtcggcggccccctagggggcgctgccagggccttggcacatggtgtccgggttctggaggacggcgtgaactatgcaacagggaacctgcccggttgctctttttctatcttcctcttggctctgctgtcctgtctgaccgtaccagcttccgctcatgaagtgcgtaacgcgtccggggtataccatgtcacgaacgactgctccaactcaagcattgtgtttgaggcggcggacttgatcatgcatactcccgggtgcgtgccctgcgttcgggagggtaactcctcccgctgctgggtagcgctcactcccacgctcgcggccaggaatgctaccatccccactacgacaatacgacaccacgtcgatttgctcgttggggcggctgctctctgctccgctatgtacgtgggggacctctgcggatctgttttcctcgtctctcagctgttcaccttctcgccccgccggcatgcgacattgcaggactgcaattgttcgatctaccccggccacgcgtcaggtcaccgcatggcctgggacatgatgatgaactggtcacctacaacagccctcgtagtgtcgcagttactccggatcccacaagccgtcatcgacatggtggcgggggcccactggggagtcctggcgggccttgcctactattccatggcggggaactgggctaaggttttgattgtgatgctactttttgccggcgttgacgggcacaccctcacaacgggggggcacgctgcccgcctcaccagcgggttcgcgggcctctttacacctgggccgtctcagagaatccagcttataaacaccaatggcagttggcacatcaacaggactgccctgaactgcaatgactccctccagactgggtttcttgccgcgctgttctacgcacataggttcaactcgtccggatgcccggagcgcatggccagctgccgctccattgacaagttcgaccagggatggggtcctatcacttatgctgagcctacaaaagacccggaccagaggccttattgctggcactacccacctcaacaatgtggtatcgtacctgcgtcgcaggtgtgtggtccagtgtattgcttcaccccaagtcctgttgtcgtggggacaaccgatcgtctcggcaaccctacgtacagctggggggagaacgatactgacgtgctgctccttaacaacacgcggccgccgcaaggcaactggttcggctgtacatggatgaatagcactgggttcaccaagacgtgcggggcccccccgtgtaacatcgggggggtcggcaataacaccttgacctgccccacggactgcttccggaagcaccccgaggccacgtactcaaaatgtggctcggggccttggttgacacctaggtgcatggttgactacccatacaggctctggcactacccctgcactgtcaacttctccatctttaaggttaggatgtatgtggggggcgtggagcacaggcttaatgctgcatgcaactggacccgaggagagcgttgcaacttggacgacagggacagatcggagctcagcccgctgctgctctctacaacagagtggcaggttctgccctgctctttcaccaccctaccggctctgtccactggcttgatccacctccatcagaacatcgtggacgtgcaatacctgtacggtatagggtcagcggttgtctcctttgcaatcaaatgggagtatgtcgtgttgcttttccttctcctggcggacgcgcgcgtctgtgcctgcttgtggatgatgctgctgatagcccaggccgaggccgccttagagaacctggtggccctcaatgcagcgtccgttgccggagcgcacggcatcctctccttcctcgtgttcttctgtgccgcttggtacatcaagggcaggctggtccctggggcggcatatgctttctatggcgcatggccgctgctcctgctcctcttgacattaccaccacgagcttacgccatggaccgggagatggctgcatcgtgcggaggcgcggtttttgtgggtctggcattattgaccttgtcgccatattacaaggtgttcctcgctaggctcctatggtggttacaatatcttatcaccagagctgaggcgcacttgcatgtgtgggttccccccctcaacgtccggggaggccgcgatgccatcatcctcctcacgtgtgcagtccacccagagctaatctttgatatcaccaaacttctgattgccatactcggaccgctcatggtgctccaagctggcataactagggtgccgtacttcgtacgcgctcaagggctcattcgtgcatgcatgttagtgcggaaagtcgctgggggtcattatgtccaaatggccttcatgagactgggcgcgctgacgggcacgtacgtctataatcacctcaccccactgcgggattgggcccacgccggcctacgggaccttgcggtagcagtggagcctgtcgtcttctctgacatggagaccaagatcatcacctggggggcagacaccgcggcgtgtggggacatcatcctgggcctacctgtctccgcccgaaggggaagggagatactcctggggccggccgatagtctagtagggcaggggtggcgactccttgcgcccatcacggcctactcccaacagacccggggcctacttggttgcatcatcacgagtctcacaggccgggacaagaaccaggtcgagggggaggttcaagtggtctccaccgcaacacaatctttcctggcgacctgcgtcaacggcgtatgttggactgtctaccatggtgctggctcaaagactctagccggcccaaaaggcccaatcgcccagatgtacactaatgtagaccaggatctcgtcggctggccggcgccccccggggcgcgttccctgacaccatgcacctgtggcagctcggacctttacttggttacgagacatgcagatgttattccggtgcgccggcggggcgacaatagagggagcttgctctcccccaggcctgtctcctacttgaagggctcttcgggtggcccactgctctgcccttcggggcacgctgtgggcgtcttccgggccgctgtatgcacccggggggttgcaaaggcggtggattttgtccccgttgagtccatggaaactactatgcggtccccggtcttcacagacaactcatctcccccggccgtaccgcaaacattccaagtggcccatctacacgctcccactggcagcggcaagagcactagagtgccggccgcatatgcggcccaagggtacaaggtgcttgtcctgaacccgtctgttgccgctaccttaggttttggggcgtatatgtctaaagcacatggtaccgaccctaacatcaggactggggtaaggaccattaccacgggcgcccccattacgtactccacctatggcaagttccttgccgacggtggttgctccgggggcgcttacgacatcataatgtgcgatgagtgccactcaactgactcaactactatcttgggcatcggcacagtcctggaccaagcggagacggctggagcgcggcttgtcgtgctcgccaccgctacgcctccaggatcggtcaccgtgccacaccccaatatcgaggaggtggccctgtcgaacactggagagatccccttctacggcaaagccatccccatcgaagccatcaaggggggaaggcacctcattttctgtcactccaagaagaagtgcgacgagcttgccgcaaagctgtcaggcctcggaatcaatgctgtagcgtattaccggggtcttgatgtgtccgtcataccgaccagcggagacgtcgttgtcgtggcaacagacgctctaatgacgggctataccggtgactttgattcagtgatcgactgtaatacgtgtgtcacccagacagtcgacttcagcttggaccccaccttcaccattgagacgacgaccgtgccccaagacgcagtgtcgcgctcgcagcggcggggtaggactggcaggggcagggggggcatatacaggtttgtaactccgggggaacggccctcgggcatgttcgattcctcggtcctgtgcgagtgctatgacgcgggctgtgcttggtacgagctcacccccgctgagacctcggttaggttgcgggcttacctaaatacaccaggattgcccgtttgccaggaccatctggagttctgggagagcgtcttcacaggcctcacccatatagatgcccacttcctgtcccagaccaagcaggcaggagataacttcccctacctggtggcataccaagccacagtgtgcgccagggctcaggccccacctccatcgtgggatcaaatgtggaagtgtctcatacggctaaaacccacgctgcacgggccaacgcccctgctgtataggctaggggccgtccaaaatgaggtcaccctcacacaccccataaccaaatacatcatggcatgcatgtcggccgacctggaagtcgtcaccagcacctgggtgctggtaggcggagtcctcgcagctctggccgcatattgcctgacaacaggcagtgtggttatcgtgggtaggatcatcttgtccgggaggccggctgtcgttcccgatagggaagtcctctaccgggagttcgatgaaatggaagaatgcgcctcgcacctcccttacatcgaacagggaatgcaactcgccgagcaattcaagcagaaggcgctcgggttgttgcaaacagccaccaagcaggcggaggctgccgctcccgtggtggagtccaagtggcgagctttggagaccttctgggcaaagcacatgtggaatttcatcagcgggatacagtacttagcgggcttatccaccctgcctgggaaccccgcgatagcatcactgatggcattcacagcctctatcaccagcccgctcaccacccagaacaccctcctgtttaacatcttgggggggtgggtagccgcccaactcgctccccccagcgctgcttcggctttcgtgggcgctggtatcgctggtgcggctgttggcagcataggtcttgggaaggtgctagtggacattctggcgggctatggggcaggggtggctggcgcgctcgtggccttcaaggtcatgagcggcgaggcgccctctgccgaggacctgatcaatttgctccctgccatcctctctcctggtgccctggtcgtcggagtcgtgtgtgcagcaatactgcgtcggcatgtgggcccgggagagggggccgtgcagtggatgaaccggctgatagcgttcgcttcgcggggtaaccatgtctcccccacgcactatgtgcctgagagcgacgccgcagcgcgtgtcactcaggtcctctccagccttaccatcacccagctgctgaagaggctccaccagtggattaatgaggactgttctacgccgtgttccggctcgtggctgagggatgtttgggactgggtgtgcacggtgttgagtgacttcaagacctggctccagtccaagctcctgccgcggttaccgggtgtccctttcctctcatgccaacgtgggtacaagggagtctggcggggggacggcatcatgcacaccacctgcccatgtggagcacagatcgccggacatgtcaaaaacggttccatgaggatcatcgggccgaaaacctgcagcaacacgtggcatggaacattccccatcaacgcgtacaccacgggcccctgcacgccttccccggcgccaaactattccaaggcgctgtggcgggtggctgctgaggagtacgtggaggtcacgcgggtgggggatttccactacgtgacgggcataaccaccgacaacgtaaagtgcccatgtcaggttccagctcctgagtttttcacggaggtggatggggtgcggttgcacaggtacgccccggtgtgcaaacctctcttacgggatgaggttgtattccaggtcgggctcaatcaatacctggttgggtcacagctcccatgcgagcccgaaccggacgtagcagtgctcacttccatgctcaccgacccctcccacattacagcagaggcggctaagcgtaggttggccagggggtctcccccctccttggccagctcttcagctagccagctgtctgcgccctccttgagggcgacatgcactacccattcttcctataatcttgactctccggacgtcgacctcattgaggccaacctcctgtggcggcaggagatgggcggaaacatcacccgcgtggagtcggagaacaaggtggtagtcctagactctttcgagccgcttcgagcggagggggatgagaatgaaatatccattgcggcggagatcctgcggaagtccaagaagttccccgcggcgatacccatatgggcacggccggattacaatcctccattgttagagtcttggaagaacccggactacgtccctccggtggtacacgggtgcccattgccacctgtcaaggcccctccaataccacctccacggagaaaaaggacggttgtcctgacggactccaccgtgtcttctgttttggcggagctcgctaccaaaaccttcggcagctccgaattgtcggccgccgacagcggcacggcgaccgcccctcctgaccagacctccgacaacggcggcaaagactccgacgctgagtcatgctcctctatgcccccccttgagggggagccgggggaccccgatctcagcgacgggtcttggtctaccgtgagcgaggaggctggtgagagcgtcgtctgctgctcaatgtcctacacatggacaggtgccctgatcacgccatgcgccgcggaagaaagcaagctgcccatcaacgcgttgagcaactctttgctgcgccatcacaacatggtctacgccacgacatcccgcagcgcgggcctgcggcagaagaaggtcacctttgacagactgcaggtcctggatgaccattaccgggacgtgcttaaggagatgaaggcaaaggcgtccacagtcaaggctaaacttctatccatagaagaagcctgccgcctgacgcccccacattcggccaaatccaagtttggctatggggcaaaggacgtccggaacctatccagcagggccatcaaccacatccgctccgtgtgggaggacttgctggaggacactgtgacaccaattgacaccaccgtcatggcaaagaatgaggttttctgcgtccaaccagagaagggaggccgcaagccagcccgccttatcgtattcccagatttgggagttcgtgtatgcgagaagatggctctctacgatgtggtctccacccttcctcaagccgtgatgggctcctcatacggattccagtactctcccgggcagcgggtcgagttcctggtaaaagcctggaaatcaaagaaaaaccctatgggcttctcatatgacacccgctgttttgactcaacggtcactgagaatgacatccgtgttgaggagtcaatttaccaatgttgtgacttggcccccgaagccagacaggctataaaatcgctcacagagcggctttatatcgggggtcccctgactaattcaaaagggcagagctgtggttatcgccggtgccgcgcgagcggcgtgctgacgactagctgcggtaataccctcacatgttacttgaaagcctctgccgcctgtcgagctgcaaagctccaggactgcacgatgctcgtgaacggggacgaccttgtcgttatctgcgaaagcgcgggaacccaggaggatgcggcgagcctacgagtcttcacggaggctatgactaggtactccgccccccccggggacttgccccaaccagaatacgacttggagttgataacatcatgttcctccaatgtgtcggtcgcgcacgatgcatctggcaaaagggtgtactacctcactcgcgatcccaccacccccatcgcacgggctgcgtgggaaacagctagacacactccagttaactcctggctaggcaacattatcatgtatgcgcccaccttatgggcaaggatgattctgatgacccatttcttctccatccttctagctcaggagcaacttgaaaaagccctggattgccaaatctacggggcctgttactccattgagccacttgacctacctcagatcattgaacgactccatggtcttagcgcattttcactccatagttactctccaggtgagatcaatagggtggcttcatgcctcaggaaacttggggtaccgcccttgcgagtctggagacatcgggccaggagcgtccgcgctaaactactgtcccagggggggagggccgccacttgcggcaaatacctcttcaactgggcagtaaagaccaagctcaaactcactccaatcccggctgcgtcccagttggacttatccggctggttcgttgctggctacagcgggggagacatatatcacagcctgtctcgtgcccgaccccgctggttcatgctgtgcctactcctactttctgtaggggtaggcatctacttgctccccaatcgatgaacggggagctaaacactccaggccaataggccatttcctgtttttttttttttttggtttttttttttttttttttttttttttttttttttttttcctttccttctttttttttttttccctctttatggtggctccatcttagccctagtcacggctagctgtgaaaggtccgtgagccgcatgactgcagagagtgctgatactggcctctctgcagatcatgt

SEQ ID NO: 11 - protein, 3015 aa, Hepatitis C virus
MSTNPKPQRKTKRNTNRRPQDVKFPGGGQIVGGVYLLPRRGPRLGVRAIRKTSERSQPRGRRQPIPKARRPEGRAWAQPGYPWPLYGNEGMGWAGWLLSPRGSRPSWGPTDPRRRSRNLGKVIDTLTCGLADLMGYIPLVGGPLGGAARALAHGVRVLEDGVNYATGNLPGCSFSIFLLALLSCLTVPASAHEVRNASGVYHVTNDCSNSSIVFEAADLIMHTPGCVPCVREGNSSRCWVALTPTLAARNATIPTTTIRHHVDLLVGAAALCSAMYVGDLCGSVFLVSQLFTFSPRRHATLQDCNCSIYPGHASGHRMAWDMMMNWSPTTALVVSQLLRIPQAVIDMVAGAHWGVLAGLAYYSMAGNWAKVLIVMLLFAGVDGHTLTTGGHAARLTSGFAGLFTPGPSQRIQLINTNGSWHINRTALNCNDSLQTGFLAALFYAHRFNSSGCPERMASCRSIDKFDQGWGPITYAEPTKDPDQRPYCWHYPPQQCGIVPASQVCGPVYCFTPSPVVVGTTDRLGNPTYSWGENDTDVLLLNNTRPPQGNWFGCTWMNSTGFTKTCGAPPCNIGGVGNNTLTCPTDCFRKHPEATYSKCGSGPWLTPRCMVDYPYRLWHYPCTVNFSIFKVRMYVGGVEHRLNAACNWTRGERCNLDDRDRSELSPLLLSTTEWQVLPCSFTTLPALSTGLIHLHQNIVDVQYLYGIGSAVVSFAIKWEYVVLLFLLLADARVCACLWMMLLIAQAEAALENLVALNAASVAGAHGILSFLVFFCAAWYIKGRLVPGAAYAFYGAWPLLLLLLTLPPRAYAMDREMAASCGGAVFVGLALLTLSPYYKVFLARLLWWLQYLITRAEAHLHVWVPPLNVRGGRDAIILLTCAVHPELIFDITKLLIAILGPLMVLQAGITRVPYFVRAQGLIRACMLVRKVAGGHYVQMAFMRLGALTGTYVYNHLTPLRDWAHAGLRDLAVAVEPVVFSDMETKIITWGADTAACGDIILGLPVSARRGREILLGPADSLVGQGWRLLAPITAYSQQTRGLLGCIITSLTGRDKNQVEGEVQVVSTATQSFLATCVNGVCWTVYHGAGSKTLAGPKGPIAQMYTNVDQDLVGWPAPPGARSLTPCTCGSSDLYLVTRHADVIPVRRRGDNRGSLLSPRPVSYLKGSSGGPLLCPSGHAVGVFRAAVCTRGVAKAVDFVPVESMETTMRSPVFTDNSSPPAVPQTFQVAHLHAPTGSGKSTRVPAAYAAQGYKVLVLNPSVAATLGFGAYMSKAHGTDPNIRTGVRTITTGAPITYSTYGKFLADGGCSGGAYDIIMCDECHSTDSTTILGIGTVLDQAETAGARLVVLATATPPGSVTVPHPNIEEVALSNTGEIPFYGKAIPIEAIKGGRHLIFCHSKKKCDELAAKLSGLGINAVAYYRGLDVSVIPTSGDVVVVATDALMTGYTGDFDSVIDCNTCVTQTVDFSLDPTFTIETTTVPQDAVSRSQRRGRTGRGRGGIYRFVTPGERPSGMFDSSVLCECYDAGCAWYELTPAETSVRLRAYLNTPGLPVCQDHLEFWESVFTGLTHIDAHFLSQTKQAGDNFPYLVAYQATVCARAQAPPPSWDQMWKCLIRLKPTLHGPTPLLYRLGAVQNEVTLTHPITKYIMACMSADLEVVTSTWVLVGGVLAALAAYCLTTGSVVIVGRIILSGRPAVVPDREVLYREFDEMEECASHLPYIEQGMQLAEQFKQKALGLLQTATKQAEAAAPVVESKWRALETFWAKHMWNFISGIQYLAGLSTLPGNPAIASLMAFTASITSPLTTQNTLLFNILGGWVAAQLAPPSAASAFVGAGIAGAAVGSIGLGKVLVDILAGYGAGVAGALVAFKVMSGEAPSAEDLINLLPAILSPGALVVGVVCAAILRRHVGPGEGAVQWMNRLIAFASRGNHVSPTHYVPESDAAARVTQVLSSLTITQLLKRLHQWINEDCSTPCSGSWLRDVWDWVCTVLSDFKTWLQSKLLPRLPGVPFLSCQRGYKGVWRGDGIMHTTCPCGAQIAGHVKNGSMRIIGPKTCSNTWHGTFPINAYTTGPCTPSPAPNYSKALWRVAAEEYVEVTRVGDFHYVTGITTDNVKCPCQVPAPEFFTEVDGVRLHRYAPVCKPLLRDEVVFQVGLNQYLVGSQLPCEPEPDVAVLTSMLTDPSHITAEAAKRRLARGSPPSLASSSASQLSAPSLRATCTTHSSYNLDSPDVDLIEANLLWRQEMGGNITRVESENKVVVLDSFEPLRAEGDENEISIAAEILRKSKKFPAAIPIWARPDYNPPLLESWKNPDYVPPVVHGCPLPPVKAPPIPPPRRKRTVVLTDSTVSSVLAELATKTFGSSELSAADSGTATAPPDQTSDNGGKDSDAESCSSMPPLEGEPGDPDLSDGSWSTVSEEAGESVVCCSMSYTWTGALITPCAAEESKLPINALSNSLLRHHNMVYATTSRSAGLRQKKVTFDRLQVLDDHYRDVLKEMKAKASTVKAKLLSIEEACRLTPPHSAKSKFGYGAKDVRNLSSRAINHIRSVWEDLLEDTVTPIDTTVMAKNEVFCVQPEKGGRKPARLIVFPDLGVRVCEKMALYDVVSTLPQAVMGSSYGFQYSPGQRVEFLVKAWKSKKNPMGFSYDTRCFDSTVTENDIRVEESIYQCCDLAPEARQAIKSLTERLYIGGPLTNSKGQSCGYRRCRASGVLTTSCGNTLTCYLKASAACRAAKLQDCTMLVNGDDLVVICESAGTQEDAASLRVFTEAMTRYSAPPGDLPQPEYDLELITSCSSNVSVAHDASGKRVYYLTRDPTTPIARAAWETARHTPVNSWLGNIIMYAPTLWARMILMTHFFSILLAQEQLEKALDCQIYGACYSIEPLDLPQIIERLHGLSAFSLHSYSPGEINRVASCLRKLGVPPLRVWRHRARSVRAKLLSQGGRAATCGKYLFNWAVKTKLKLTPIPAASQLDLSGWFVAGYSGGDIYHSLSRARPRWFMLCLLLLSVGVGIYLLPNR

SEQ ID NO: 12 - protein, 191 aa, Hepatitis C virus
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAAS

SEQ ID NO: 13 - protein, 191 aa, Hepatitis C virus
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPFVGAPVGGVARALAHGVRALEDGINFATGNLPGCSFSIFLLALFSCLVHPAAS

SEQ ID NO: 14 - protein, 191 aa, Hepatitis C virus
MSTLPKPQRKTKRNTIRRPQDVKFPGGGQIVGGVYVLPRRGPRLGVRTTRKTSERSQPRGRRQPIPKARRSEGRSWAQPGFPWPLYGNEGCGWAGWLLSPRGSRPSWGPNDPRRRSRNLGKVIDTLTCGFADLMGYIPLVGAPVGGVARALAHGVRALEDGINFATGNLPGCSFSVFLLALLSCLIHPAAS

SEQ ID NO: 15 - protein, 191 aa, Hepatitis C virus
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALLSCLIHPAAS

SEQ ID NO: 16 - protein, 191 aa, Hepatitis C virus
MSTLPKPQRKTKRNTIRRPQDVKFPGGGQIVGGVYVLPRRGPRLGVRAARKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAAS

(19) United States
(12) Patent Application Publication
(10) Pub. No.: US 2009/0252745 A1
(43) Pub. Date: Oct. 8, 2009

(54) Amino acids in the HCV core polypeptide domain 3 and correlation with steatosis

(75) Inventors: Ravi R. Jhaveri, Durham, NC (US); John G. McHutchison, Chapel Hill, NC (US); Keyur Patel, Chapel Hill, NC (US); Anna Mae Diehl, Durham, NC (US)

Correspondence Address: JENKINS, WILSON, TAYLOR & HUNT, P. A., Suite 1200 UNIVERSITY TOWER, 3100 TOWER BLVD.,, DURHAM, NC, 27707, US

(73) Assignee: Duke University, Durham, NC (US)

(21) Appl. No.: 11/901,324

(22) Filed: Sep. 17, 2007

Related U.S. Application Data
(60) Provisional application No. 60/845,078, filed on Sep. 15, 2006.

(51) Int. Cl. C12Q 1/70; C40B 30/06; A61K 39/42

(52) U.S. Cl. 424/159.1; 435/5; 506/10; 530/387.1

(57) ABSTRACT

The presently disclosed subject matter provides methods and compositions for predicting a tendency of a subject infected with hepatitis C virus (HCV) to develop steatosis. In some embodiments, the disclosed methods include the steps of (a) isolating from the subject a biological sample comprising an HCV Core polypeptide or a nucleic acid molecule encoding an HCV Core polypeptide; and (b) identifying the amino acids in the HCV Core polypeptide or encoded by the nucleic acid molecule in the biological sample corresponding to positions 182/186 of an HCV Core polypeptide amino acid sequence, whereby a tendency to develop steatosis in the subject is predicted when the amino acids corresponding to positions 182/186 of the HCV Core polypeptide amino acid sequence in the biological sample are either phenylalanine/valine or leucine/isoleucine. Also provided are compositions and methods for screening for candidate modulators of lipid accumulation in a subject as well as uses for the candidate modulators.

### CROSS REFERENCE TO RELATED APPLICATION

The presently disclosed subject matter claims the benefit of U.S. Provisional Patent Application Ser. No. 60/845,078, filed Sep. 15, 2006; the disclosure of which is incorporated herein by reference in its entirety.

### GRANT STATEMENT

The presently disclosed subject matter was made with United States Government support under Grant No. K12HD043494 from the National Institutes of Health and Grant No. HD043494 awarded by the National Institutes of Health—National Institute of Child Health and Human Development. Thus, the United States Government has certain rights in the presently disclosed subject matter.

### TECHNICAL FIELD

The presently disclosed subject matter generally relates to the field of viral diseases. More particularly, the presently disclosed subject matter relates to methods and compositions for predicting a tendency of a subject infected with hepatitis C virus (HCV) to develop steatosis. Also provided are methods and compositions for modulating lipid accumulation in a subject.

### BACKGROUND

Hepatitis C virus (HCV) is a major public health concern with almost 3 million Americans having chronic infection. In the United States, HCV infection is currently the leading indication for adult liver transplant, causes 8,000-10,000 deaths per year, and has projected costs to society of $20-50 billion for the decade 2010-19 (Wong et al., 2000).

HCV is a single stranded, plus sense RNA virus. It is classified within the Flavivirus family in its own Hepacivirus genus. The genome is approximately 9600 base pairs and is organized as one long open reading frame (ORF) flanked by 5′ and 3′ untranslated regions (Forms & Bukh, 1999; Regev & Schiff, 2000). Genes that encode the structural proteins of the virus (Core, E1, and E2) are toward the 5′ end of the genome, while genes that encode the non-structural proteins (p7, NS2, NS3, NS4A, NS4B, NS5A, and NS5B) are located in the 3′ end of the genome.

There are 6 different major genotypes of HCV. In the United States, genotypes 1a and 1b account for 70-90% of the clinical isolates (Regev & Schiff, 2000). Genotype 3 accounts for 5-10% in the US but is more prevalent is Europe and Australia. Also, genotype 3 may be more prevalent in certain isolated populations, including injection drug users (Forms & Bukh, 1999; Regev & Schiff, 2000).

Steatosis, or fat accumulation within hepatocytes, is often seen on liver histology in HCV infected patients. Since the initial observations, a number of primarily retrospective studies have examined the relationship between steatosis and HCV induced liver disease and concluded that steatosis is an independent factor associated with accelerated fibrosis progression and an impaired response to interferon-based therapy (Poynard et al., 2003). Recent studies have estimated that approximately 50% of patients with chronic HCV infection have some evidence of steatosis on liver biopsy (Patton et al., 2004). 80% of patients with genotype 3 infection have evidence of steatosis versus 30-40% of those with genotype 1 infection (Ramalho, 2003).

The pathogenesis of steatosis appears to be multifactorial. Host factors that appear to be important include alcohol use, obesity, diabetes, insulin resistance, and leptin levels (Ramalho, 2003). Several studies have shown patients with chronic HCV genotype 3 infection have steatosis on biopsy that closely correlates with serum viral load and resolves with successful therapy, suggesting a viral etiology that is independent of the previously mentioned host factors (Patton et al., 2004; Ramalho, 2003; Romero-Gomez et al., 2003; Hezode et al., 2004). Steatosis that is observed in other genotypes seems to more closely correlate with host and environmental factors (Patton et al., 2004; Hezode et al., 2004; Lonardo et al., 2004).

What are needed, then, are tools to predict subpopulations of HCV-infected subjects that are likely to develop steatosis, and/or are prone to more extreme cases of steatosis. Also needed are new compositions that can be employed for modulating steatosis in HCV-infected subjects. The presently disclosed subject matter addresses these and other needs in the art.

### SUMMARY

The presently disclosed subject matter provides methods for predicting a tendency to develop steatosis in a subject infected with hepatitis C virus (HCV). In some embodiments, the presently disclosed methods comprise (a) isolating from the subject a biological sample comprising an HCV Core polypeptide and/or a nucleic acid molecule encoding an HCV Core polypeptide; and (b) identifying the amino acids in the HCV Core polypeptide and/or encoded by the nucleic acid molecule in the biological sample corresponding to positions 182/186 of SEQ ID NO: 2, whereby a tendency to develop steatosis in the subject is predicted when the amino acids corresponding to positions 182/186 of SEQ ID NO: 2 in the biological sample are either phenylalanine/valine or leucine/isoleucine.

In some embodiments, the biological sample is selected from the group consisting of a blood sample or a biopsy. In some embodiments, the biological sample comprises an HCV virion, an HCV genomic RNA molecule, or an RNA molecule encoded by an HCV genomic RNA molecule. In some embodiments, the identifying is by nucleic acid sequencing and/or amino acid sequencing, and in some embodiments the identifying is by contacting the biological sample with an antibody that differentiates between a HCV Core polypeptide that has an FV or an LI amino acid pair at positions corresponding to amino acids 182/186 of SEQ ID NO: 2 and an HCV Core polypeptide that does not have an FV or an LI amino acid pair at positions corresponding to amino acids 182/186 of SEQ ID NO: 2. In some embodiments, the contacting is performed with the HCV Core polypeptide in solution in the biological sample, and in some embodiments the contacting is performed subsequent to transferring the HCV Core polypeptide to a solid support. In some embodiments, the antibody comprises a detectable label comprising a moiety selected from the group consisting of a light-absorbing dye, a fluorescent dye, a radioactive label, an enzyme, an epitope tag, and biotin.

The presently disclosed subject matter also provides methods for screening for a candidate molecule that modulates lipid accumulation in a cell. In some embodiments, the presently disclosed methods comprise (a) providing a cell infected with HCV, wherein the HCV present therein encodes a Core polypeptide comprising an FV or an LI amino acid pair at positions corresponding to amino acids 182/186 of SEQ ID NO: 2; (b) contacting the cell with a candidate molecule under conditions sufficient to allow the candidate molecule to interact with the HCV Core polypeptide and/or to interact with a molecule that interacts with the HCV Core polypeptide in the cell; (c) quantifying lipid accumulation in the cell; and (d) comparing lipid accumulation in the cell in the presence of the candidate molecule to lipid accumulation in the cell in the absence of the candidate molecule.

In some embodiments, the candidate molecule is provided in the form of a library. In some embodiments, the library comprises ten or more diverse molecules, in some embodiments one hundred or more diverse molecules, and in some embodiments a billion or more diverse molecules. In some embodiments, the library of diverse molecules comprises a library of molecules selected from the group consisting of peptides, peptide mimetics, proteins, antibodies and/or fragments and/or derivatives thereof, small molecules, nucleic acids, and combinations thereof. In some embodiments, the library of diverse molecules comprises a library of peptides, antibodies and/or fragments and/or derivatives thereof, small molecules, or a combination thereof.

The presently disclosed subject matter also provides a molecule identified by the disclosed screening methods.

The presently disclosed subject matter also provides methods for modulating lipid accumulation in a cell. In some embodiments, the cell is present in a subject. In some embodiments, the presently disclosed methods comprise administering a therapeutically effective amount of a composition comprising a molecule identified by the presently disclosed screening methods. In some embodiments, the lipid accumulation is associated with steatosis. In some embodiments, the steatosis comprises lipid accumulation in the liver of the subject. In some embodiments, the steatosis is incident to infection with HCV. In some embodiments, the administering is by a route selected from the group consisting of oral, intravenous, intramuscular, transdermal, and inhalation.

In some embodiments of the presently disclosed methods, the subject is a mammal. In some embodiments, the mammal is a human. In some embodiments, the human is infected with HCV.

Accordingly, it is an object of the presently disclosed subject matter to provide a method for predicting a tendency of a subject infected with hepatitis C virus (HCV) to develop steatosis. This and other objects are achieved in whole or in part by the presently disclosed subject matter.

An object of the presently disclosed subject matter having been stated above, other objects and advantages of the presently disclosed subject matter will become apparent to those of ordinary skill in the art after a study of the following description, Figures, and non-limiting Examples.

### BRIEF DESCRIPTION OF THE DRAWINGS

The instant application file contains at least one drawing executed in color. Copies of this patent or patent application publication with color drawing(s) will be provided by the United States Patent and Trademark Office upon request and payment of the necessary fee.

FIGS. 1A-1C depict the results of amplification and sequence analysis of HCV Core clones.

FIG. 1A shows the deduced amino acid sequences of eight (8) different HCV Core gene clones and a consensus sequence (CON_{—}3a; SEQ ID NO: 12) derived from sequencing the amplified nucleic acids that correspond to each HCV Core clone. The amino acid sequences listed are for clones HCV1 (SEQ ID NO: 13); HCV12 (SEQ ID NO: 14); HCV17 (SEQ ID NO: 15); HCV26 (SEQ ID NO: 15); HCV3 (SEQ ID NO: 12); HCV11 (SEQ ID NO: 12); HCV16 (SEQ ID NO: 16); and HCV23 (SEQ ID NO: 12).

FIG. 1B depicts an agarose gel showing RT-PCR amplified fragments of representative HCV Core gene. RT-PCR products were run along with a negative control that lacked any template RNA. The amplified HCV Core products all ran below the marker band corresponding to 650 bp.

FIG. 1C depicts a comparison of predicted amino acid sequences within domain 3 of HCV Core using the ClustalW (EMBL-EBI) program. The sample numbers correspond to those assigned as part of the study. The corresponding sequences are aligned to the right and show amino acids 181-190 of the Core polypeptide sequence. Samples from patient 1, 17, 26, and 12 are from patients with steatosis on biopsy and appear on top. The amino acid pairs in positions 182 and 186 are phenylalanine-valine (FV) or leucine-isoleucine (LI) from their samples and are in bold and underlined. Three of four patients with steatosis had the LI pair in those positions. The samples from patients 11, 16, and 23 are from patients without steatosis on biopsy and appear on the bottom. All four patients had the amino acid pair phenylalanine-isoleucine (FI) in their samples in amino acid positions 182 and 186 and these are in bold and underlined. The samples from patient 3 yielded the only discordant result. The correlation of steatosis with the LI pair of amino acids was significant (p=0.028) as was the correlation of no steatosis with the FI pair (p=0.005).

FIG. 2 depicts a Western blot confirming protein expression of cloned HCV genes. Protein lysates were prepared 72 hours after transfection of Huh-7 cells with a plasmid containing HCV Core cDNA clones. The following samples are depicted on this gel: HCV core positive control (+cont), GFP plasmid (GFP), empty vector (EV), three clones of HCV Core polypeptide (HCV1, HCV11, and HCV12), and their corresponding mutated clones (m). Each clone is labeled with its amino acid pair at positions 182 and 186: FV, LI, or FI.

FIGS. 3A and 3B depict fluorescent and brightfield microscopy of HCV Core expressing cells.

FIG. 3A depicts overlaid images from HepG2 cells after immunofluorescence (IF) and Oil Red 0 (ORO) staining. Panel 1 depicts anti-HCV Core (green) and DAPI (blue) staining. Panel 2 depicts a brightfield view of ORO stain to assess intracellular lipid content (red). Panel 3 depicts Panels 1 and 2 overlaid. Cells that express HCV Core polypeptide clone possess high amounts of intracellular fat. All images are 100× magnification.

FIG. 3B depicts overlaid images from 5H cells after HCV Core and Oil Red 0 staining. Panel 1 depicts anti-HCV Core (green) and DAPI (blue) staining. Panel 2 depicts a brightfield view of ORO stain to assess intracellular lipid content (red). Panel 3 depicts Panels 1 and 2 overlaid. 5H cells expressing the HCV Core clone possess more intracellular fat than cells the do not express the protein. All images are 68× magnification.

FIGS. 4A-4F depict images that offer an overview of analysis using the METAMORPH® system (Molecular Devices Corp., Downingtown, Pa., United States of America). Image files corresponding to the IF and ORO views were opened within the program. Areas of green on the IF images were visualized and a region was designed that corresponded to the HCV Core expressing cell(s) (FIG. 4A). After a green color threshold was applied (FIG. 4B) and the data was recorded, these regions were then transferred to the corresponding brightfield image at the same coordinates on the image (FIG. 4C). A red color threshold was applied to the brightfield image (FIG. 4D) and then the percent of the region that met or exceeded the red threshold was recorded. This number corresponded to the percent of the cell stained with Oil Red 0 that appeared red in the photograph. For the example image, 9.44% of the image met or surpassed the Red Threshold.

FIGS. 4E and 4F depict images from experiments validating the METAMORPH® system analysis of ORO staining. In FIG. 4E, 63× images of 5H cells incubated in media containing 2% FBS, 10% FBS and 20% FBS fixed and stained with ORO. The images show that intracellular lipid increases with increasing FBS concentration. FIG. 4F is a bar graph depicting the results of analyzing ten (10) 40× images from each slide well depicting in FIG. 4E using METAMORPH® software.

FIGS. 5A-5D depict METAMORPH® analysis of ORO staining of cells containing various HCV clones. Twenty 68× high power fields were compared for each of the clones represented. As set forth hereinabove with reference to FIG. 4, results are expressed as percent of ORO stain as measured by area within the region that met or surpassed the red threshold applied.

FIG. 5A is a graph of Oil Red staining of cells containing HCV1, HVC11, HCV12, or HCV-N (see FIG. 1). 5H cells expressing the GFP control vector had an average of 1.1% of their region area stain with ORO. Cells expressing the HCV1 clone (steatosis) had an average of 11.4%, as opposed to cells expressing the HCV11 clone (non-steatosis) which had only 7.8% of the region stain with ORO. This difference was significant (p=0.02). Cells expressing HCV12 (steatosis) had an average of 10.8%, which was also significant over HCV11 (p=0.01).

FIG. 5B depicts IF and ORO images are depicted for cells containing a GFP vector (negative control), HCV1, or HCV11.

FIG. 5C is a graph of HCV1 vs. HCV1 V1861 mutant staining. After the HCV1 clone had its amino acid at position 186 changed from valine to isoleucine (HCV mut), which should change its phenotype from steatosis to non-steatosis, cells expressing this HCV1 mutant clone had an average of only 8.3% of their region stain with ORO. When compared with the parent HCV1 clone, this difference was significant (p=0.03).

FIG. 5D depicts images from each group that represent average values for % ORO stain in METAMORPH® analysis. IF and ORO images are depicted for cells containing a GFP vector (negative control), HCV1, and HCV1 V1861 (HCV1 mut).

FIGS. 6A-6F depict the results of experiments using fusion proteins of green fluorescent protein (GFP) with various Domains of HCV Core Protein.

FIG. 6A depicts various GFP-HCV Core Protein (amino acids 1-191) fusion constructs. The Figure shows the locations of Domain 1 (amino acids, 1-117), Domain 2 (amino acids 118-178), and Domain 3 (amino acids 179-191), and fusion constructs that fused GFP to the full length HCV Core Protein, to Domains 2 and 3 (i.e., to amino acid 118 of the HCV Core Protein, deleting amino acids 1-117), and to Domain 3 (i.e., to amino acid 179 of the HCV Core Protein, deleting amino acids 1-178).

FIGS. 6B-6D depict a series of photographs of cells expressing HCV Core deletion mutants fused to GFP. FIG. 6B depicts a comparison of stable cells expressing GFP alone compared to cells expressing GFP fused to Domains 2 and 3 of HCV Core. Note the lipid aggregates present in the Domain 2-3 expressing cells. FIG. 6C depicts a brightfield microscope photograph of cells expressing GFP fused to Domain 3 alone. Note the multiple, large cytoplasmic vacuoles present in almost all cells. FIG. 6D depicts Oil Red 0 staining of cells expressing Domain 3 alone. Note the overlap of large vacuoles on the fluorescent image with the large lipid containing vacuoles in the brightfield image.

FIG. 6E is a bar graph depicting the results of METAMORPH® analysis of stable cells expressing Core deletion constructs. FIG. 6E shows an increased amount of Oil Red 0 stain in cells expressing GFP-Core deletion constructs. Cells expressing Domain 3 alone had the highest amount of intracellular lipid (26%), which was significantly higher than cells expressing Domain 2-3 or GFP alone (p<0.00001 for both). Cells expressing Domain 2-3 had significantly more lipid than cells with GFP alone (p=0.002).

FIG. 6F is a bar graph depicting triglyceride content analysis of stable cells expressing Core deletion constructs. FIG. 6F shows an increased amount of triglycerides per 100 μg of total protein in cells expressing GFP-Core deletion constructs. Cells expressing Domain 3 alone had the highest triglycerides level (12.4%), which was significantly higher than cells expressing Domain 2-3 or 5H control cells (p=0.01 for both). Cells expressing Domain 2-3 also had significantly more lipid than control cells (p=0.02).

### BRIEF DESCRIPTION OF THE SEQUENCE LISTING

SEQ ID NO: 1 is a nucleic acid sequence of a representative HCV genome. It corresponds to GENBANK® Accession No. D17763 (Hepatitis C virus (isolate NZL1) genomic RNA, complete genome).

SEQ ID NO: 2 is the amino acid sequence set forth in GENBANK® Accession No. BAA04609, which corresponds to the amino acid sequence of the HCV polyprotein encoded by nucleotides 340 to 9405 of SEQ ID NO: 1. Amino acids 1-191 of GENBANK® Accession No. BAA04609, which are referred to in the annotations therein as the “C protein”, correspond to the Core protein as referred to herein. It also corresponds to amino acids 1-191 of SEQ ID NO: 2 and is encoded by nucleotides 685-909 of SEQ ID NO: 1. Amino acids 182 and 186 are encoded by nucleotides 883-885 and 895-897, respectively, of SEQ ID NO: 1.

SEQ ID NO: 3 is the nucleotide sequence of an artificially synthesized oligonucleotide primer that can be employed along with SEQ ID NO: 4 to amplify a nucleic acid sequence that encodes an HCV Core Genotype 3 amino acid sequence. Nucleotides 17-34 of SEQ ID NO: 3 are identical to nucleotides 341-357 of SEQ ID NO: 1.

SEQ ID NO: 4 is the nucleotide sequence of an artificially synthesized oligonucleotide primer that can be employed along with SEQ ID NO: 3 to amplify a nucleic acid sequence that encodes an HCV Core Genotype 3 amino acid sequence.

SEQ ID NO: 5 is the nucleotide sequence of an artificially synthesized oligonucleotide primer that includes an isoleucine codon at nucleotides 895-897 of SEQ ID NO: 1, which corresponds to amino acid 186 of SEQ ID NO: 2.

SEQ ID NO: 6 is the nucleotide sequence of an artificially synthesized oligonucleotide primer that can be used to mutagenize the isoleucine codon encoded by nucleotides 895-897 of SEQ ID NO: 1, which corresponds to amino acid 186 of SEQ ID NO: 2, to a valine by changing position 895 from an A to a G.

SEQ ID NO: 7 is the nucleotide sequence of an artificially synthesized oligonucleotide primer that that includes a phenylalanine codon at nucleotides 883-885 and an isoleucine codon at nucleotides 895-897 of SEQ ID NO: 1, which correspond to amino acids 182 and 186 of SEQ ID NO: 2, respectively.

SEQ ID NO: 8 is the nucleotide sequence of an artificially synthesized oligonucleotide primer that can be used to sequence a nucleic acid comprising a CMV immediate early-1 (IE-1) gene promoter sequence.

SEQ ID NO: 9 is the nucleotide sequence of an artificially synthesized oligonucleotide primer that can be used to sequence a nucleic acid comprising a simian virus 40 (SV40) polyadenylation signal nucleotide sequence.

SEQ ID NO: 10 is a nucleic acid sequence of a representative HCV genome. It corresponds to GENBANK® Accession No. AF139594 (Hepatitis C virus strain HCV-N, complete genome).

SEQ ID NO: 11 is the amino acid sequence set forth in GENBANK® Accession No. AAD44718, which corresponds to the amino acid sequence of the HCV polyprotein encoded by nucleotides 342 to 9389 of SEQ ID NO: 9. As set forth in the annotations to GENBANK Accession No. AAD44718, amino acids 116-190 of SEQ ID NO: 11 correspond to the HCV Core protein, which is encoded by nucleotides 687-911 of SEQ ID NO: 10. Amino acids 182 and 186 are encoded by nucleotides 885-887 and 897-899, respectively, of SEQ ID NO: 10.

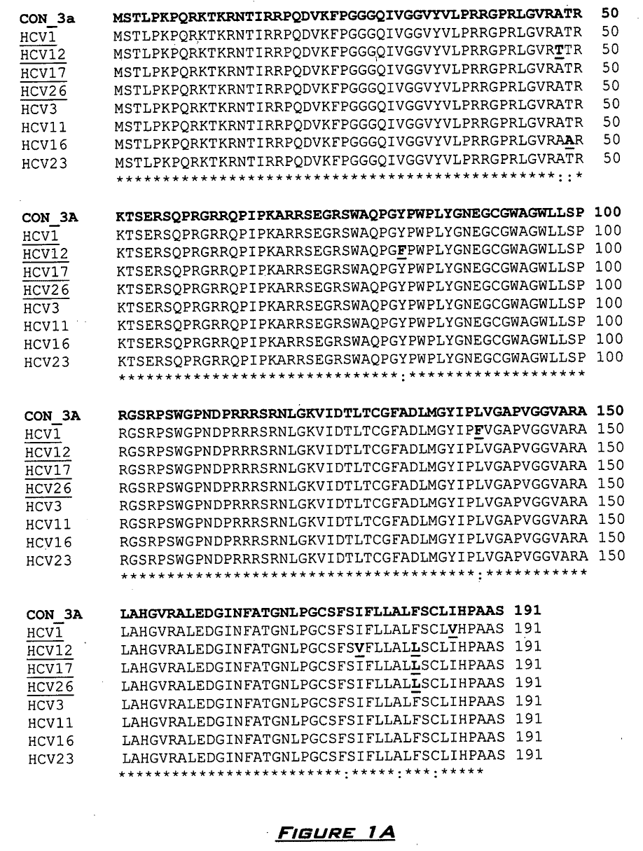

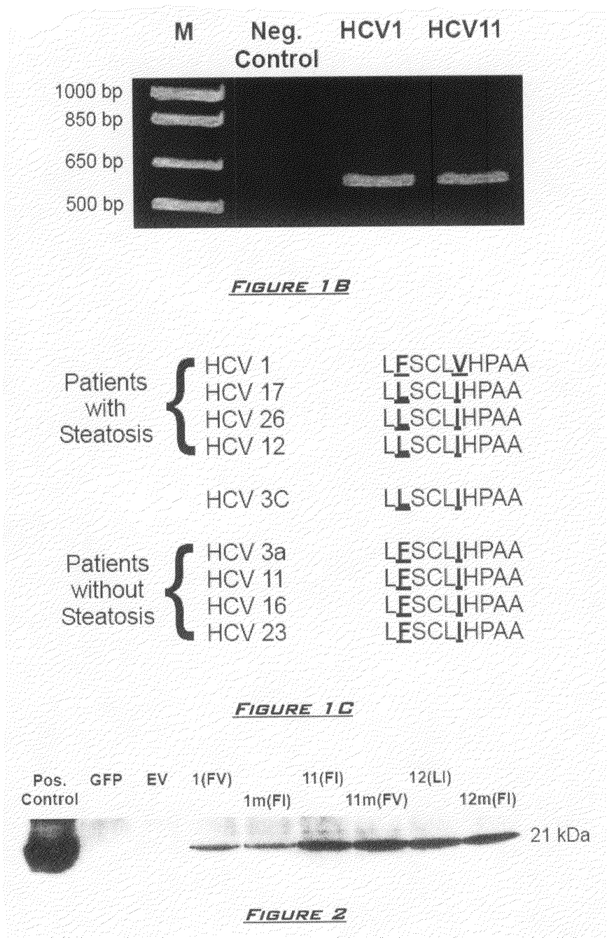

SEQ ID NOs: 12-16 are the amino acid sequences derived from sequencing amplified nucleic acids from several HCV Core gene isolates. SEQ ID NO: 12 corresponds to the deduced amino acid sequence for isolates HCV3, HCV11, HCV23, and the consensus sequence shown in FIG. 1A (CON_{—}3a). SEQ ID NO: 13 corresponds to the deduced amino acid sequence for isolate HCV1. SEQ ID NO: 14 corresponds to the deduced amino acid sequence for isolate HCV12. SEQ ID NO: 15 corresponds to the deduced amino acid sequence for isolates HCV17 and HCV26. SEQ ID NO: 16 corresponds to the deduced amino acid sequence for isolate HCV16.

### DETAILED DESCRIPTION

### 1. General Considerations

Previous work on steatosis and in vitro expression of individual HCV proteins, mostly with Core polypeptide, has been with genotype 1 isolates. This work has shown that HCV Core transgenic mice inconsistently developed steatosis, that Core and NS5A co-localize to lipid droplets within hepatoma cells, and that Core inhibits triglyceride transfer and VLDL synthesis (Moriya et al., 1997; Shi et al., 2002; Perlemuter et al., 2002).

Given that steatosis is considerably more frequently encountered in subjected infected with HCV genotype 3 than in HCV genotype 1, it was considered whether sequence differences between HCV genotypes 1 and 3 might correlate with clinical development of steatosis. It was further considered whether expression of genes encoding polypeptides comprising amino acid sequence differences between genotypes 1 and 3 might alter lipid metabolism within the liver in a medically relevant way in HCV-infected subjects. Disclosed herein is a showing that specific amino acid pairs within the terminal domain of the HCV Core polypeptide do in fact correlate with clinical steatosis.

### II. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, for example, reference to “a vector” includes a plurality of such vectors, and so forth. Similarly, reference to “a cell” includes a plurality of cells, and in some embodiments can include a tissue and/or an organ.

As used herein, the term “about,” when referring to a value or to an amount of mass, weight, time, volume, concentration or percentage is meant to encompass variations of in some embodiments±20%, in some embodiments±10%, in some embodiments±5%, in some embodiments±1%, and in some embodiments±0.1% from the specified amount, as such variations are appropriate to perform the disclosed method.

As used herein, “significance” or “significant” relates to a statistical analysis of the probability that there is a non-random association between two or more entities. To determine whether or not a relationship is “significant” or has “significance”, statistical manipulations of the data can be performed to calculate a probability, expressed as a “p-value”. Those p-values that fall below a user-defined cutoff point are regarded as significant. A p-value in some embodiments less than or equal to 0.1, in some embodiments less than 0.05, in some embodiments less than 0.01, in some embodiments less than 0.005, and in some embodiments less than 0.001 are regarded as significant.

### III. Predictive and/or Prognostic Methods

The presently disclosed subject matter provides in some embodiments methods for predicting a tendency to develop steatosis in a subject infected with hepatitis C virus (HCV). In some embodiments, the method comprises (a) isolating from the subject a biological sample comprising an HCV Core polypeptide or a nucleic acid molecule encoding an HCV Core polypeptide; and (b) identifying the amino acids in the HCV Core polypeptide or encoded by the nucleic acid molecule in the biological sample corresponding to positions 182/186 of SEQ ID NO: 2, whereby a tendency to develop steatosis in the subject is predicted when the amino acids corresponding to positions 182/186 of SEQ ID NO: 2 in the biological sample are either phenylalanine/valine or leucine/isoleucine. Information concerning the genotype of the HCV present in the subject can be employed, for example, to inform a physician as to whether or not additional and/or more aggressive therapies should be considered for the subject (e.g., therapies designed to modulate the development of steatosis in those subjects that are at increased risk of developing steatosis based on the genotype of the HCV).

SEQ ID NO: 2 discloses a representative amino acid sequence for the HCV polyprotein. One of ordinary skill in the art would understand, however, upon review of the instant disclosure that HCV isolates can differ in the amino acid sequence of the polyprotein, and by extension, the amino acid sequences of various proteins that are fragments of the polyprotein. Accordingly, it is understood that to be predictive of a tendency to develop steatosis, changes other than at positions 182 and 186 of SEQ ID NO: 2 might or might not be informative.

It is further understood that in some embodiments and as disclosed herein, consideration of the amino acids that are present both at position 182 and at position 186 is important. For example and as set forth in more detail herein below, certain isolates of HCV are characterized by a phenylalanine (F) residue at position 182, while others have a leucine (L) at this position. Certain isolates are also characterized by a valine (V) at position 186, while others have an isoleucine (I). Thus, there are four (4) different possibilities at these positions: FV, FI, LV, and LI that involve only these amino acids (although there are actually 400 different potential amino acid “pairs” at these positions). As disclosed herein, the pairs FV and LI were associated with steatosis, whereas the pair FI was not (the pair LV was not observed). Accordingly, consideration of the amino acid only at either position 182 or at position 186 can be uninformative.

In some embodiments of the presently disclosed methods, the amino acid sequence of an HCV Core polypeptide (or a fragment thereof) is determined. In some embodiments, the presently disclosed methods are employed using a biological sample isolated from a subject. As used herein, the phrase “biological sample” refers to any sample (e.g., a cell, tissue, and/or fluid) that can be isolated from a subject and assayed for the presence of an HCV virus and/or for the presence of a biomolecule indicative of HCV infection. As used herein, the phrase “biomolecule indicative of HCV infection” refers to a biomolecule (e.g., a nucleic acid or a polypeptide) that is detectable in a subject that has been infected by HCV but that is not detectable in (e.g., is absent from) a subject that has not been infected with HCV. The biomolecule indicative of HCV infection can thus comprise, for example, an HCV virion, an HCV genomic RNA molecule, an RNA molecule encoded by an HCV genomic RNA molecule, or combinations thereof.

Any biological sample can be employed to assay for the presence of a biomolecule indicative of HCV infection. For example, in some embodiments the biological sample is selected from the group including, but not limited to, a blood sample or a biopsy. Other tissues, cells, and/or biological fluids that might be expected to comprise a biomolecule indicative of HCV infection are known in the art.

Methods for assaying a biological sample for the presence of a biomolecule indicative of HCV infection are also known in the art, and can include various techniques depending on the type of molecule being assayed. For example, in some embodiments the biomolecule indicative of HCV infection comprises a nucleic acid molecule, and the identifying is by nucleic acid sequencing with or without amplification of one or more of the nucleic acids present in the biological sample.

Thus, in some embodiments the amino acids at positions that correspond to amino acids 182 and 186 of SEQ ID NO: 2 in a given biological sample are confirmed by isolating a nucleic acid molecule indicative of HCV infection from the biological sample or from another site in the subject and sequencing the nucleic acid molecule to determine which amino acids are present in these positions. In the case of a DNA molecule, one of ordinary skill in the art can design one or more primers that can be used to amplify and/or sequence one or more DNA molecules present in or isolated from a biological sample based on the published sequences of the HCV genome and the products encoded thereby. An exemplary genomic sequence is provided in GENBANK® Accession No. D17763. Techniques for designing primers and isolating DNA (and amplifying the same, if desired) are known in the art. In some embodiments, the nucleic acid is an RNA molecule, which can be sequenced directly or reverse transcribed (with or without amplification) prior to sequencing, if desired.

Additionally, allele-specific primers can also be employed to genotype and/or amplify biomolecules indicative of HCV infection using techniques known to the skilled artisan. As used herein, the phrase “allele-specific primer” refers to a primer that binds to a nucleic acid molecule that includes a specific codon at a position encoding an amino acid corresponding to amino acid 182 and/or amino acid 186 of SEQ ID NO: 2, but that does not bind to a nucleic acid molecule that includes a different codon at a position encoding an amino acid corresponding to amino acid 182 and/or amino acid 186 of SEQ ID NO: 2. Stated another way, employing allele-specific primers can be used to distinguish between HCV isolates that encode different Core polypeptides because only certain allele-specific primers will successfully amplify a given Core gene sequence. One of ordinary skill in the art understands how to design appropriate primers to distinguish between different Core gene sequences. A non-limiting example of how this can be accomplished is to design a primer that has as its 3′ terminal nucleotide sequence a sequence that is 100% complementary to a codon that encodes amino acid 182 or 186 of an HCV Core polypeptide, since it is known that the polymerase chain reaction (PCR) is particularly sensitive to mismatches at the 3′ ends of primers.

In the case where the biomolecule is a polypeptide, techniques for isolating and/or purifying and/or sequencing polypeptides are also known. Generally, however, the isolating of a polypeptide using a specific antibody is sufficient evidence of HCV infection, and sequencing might not be necessary to identify the presence of the biomolecule in the sample (although sequencing might be required to identify the amino acids at positions that correspond to amino acids 182 and 186 of SEQ ID NO: 2). Alternatively or in addition, antibodies can be generated and employed that specifically bind to HCV Core polypeptides that have particular combinations of amino acids at positions 182 and 186 of an HCV Core polypeptide using techniques that are well known in the art. See e.g., Harlow & Lane, 1988.

For example, different antibodies can be produced that distinguish between HCV isolates that have the FV or the LI pair at positions 182/186 versus HCV isolates that have some other combination of amino acids at these positions. Thus, in some embodiments the identifying is by contacting the biological sample with an antibody that differentiates between a HCV Core polypeptide that has an FV or an LI amino acid pair at positions corresponding to amino acids 182/186 of SEQ ID NO: 2 and an HCV Core polypeptide that does not have an FV or an LI amino acid pair at positions corresponding to amino acids 182/186 of SEQ ID NO: 2. In some embodiments, the contacting is performed with the HCV Core polypeptide in solution in the biological sample, while in some embodiments the contacting is performed subsequent to transferring the HCV Core polypeptide to a solid support. In some embodiments, the antibody comprises a detectable label comprising a moiety selected from the group consisting of a light-absorbing dye, a fluorescent dye, a radioactive label, an enzyme, an epitope tag, and biotin.

### IV. Screening Methods

IV.A. Candidate Molecules

The presently disclosed subject matter also provides methods for screening for a candidate molecule that modulates lipid accumulation in a cell. In some embodiments, the method comprises (a) providing a cell infected with HCV, wherein the HCV present therein encodes a Core polypeptide comprising an FV or an LI amino acid pair at positions corresponding to amino acids 182/186 of SEQ ID NO: 2; (b) contacting the cell with a candidate molecule under conditions sufficient to allow the candidate molecule to interact with the HCV Core polypeptide and/or to interact with a molecule that interacts with the HCV Core polypeptide in the cell; (c) quantifying lipid accumulation in the cell; and (d) comparing lipid accumulation in the cell in the presence of the candidate molecule to lipid accumulation in the cell in the absence of the candidate molecule.

In some embodiments, the candidate molecule is provided in the form of a library. As used herein, the term “library” means a collection of molecules. A library can contain a few or a large number of different molecules, varying from at least two molecules to several billion molecules or more. A molecule can comprise a naturally occurring molecule, or a synthetic molecule that is not found in nature. Optionally, a plurality of different libraries can be employed simultaneously for in vivo and/or in vitro screening.

Representative libraries include but are not limited to a peptide library (U.S. Pat. Nos. 6,156,511; 6,107,059; 5,922,545; and 5,223,409), an oligomer library (U.S. Pat. Nos. 5,650,489 and 5,858,670), an aptamer library (U.S. Pat. Nos. 6,180,348 and 5,756,291), a small molecule library (U.S. Pat. Nos. 6,168,912 and 5,738,996), a library of antibodies or antibody fragments (for example, an scFv library or an Fab antibody library; U.S. Pat. Nos. 6,174,708; 6,057,098; 5,922,254; 5,840,479; 5,780,225; 5,702,892; and 5,667,988), a library of nucleic acid-protein fusions (U.S. Pat. No. 6,214,553), and a library of any other affinity agent that can potentially bind to an HCV Core polypeptide (e.g., U.S. Pat. Nos. 5,948,635; 5,747,334; and 5,498,538). In some embodiments, a library is a phage-displayed antibody library. In some embodiments, a library is a phage-displayed scFv library. In some embodiments, a library is a phage-displayed Fab library. In some embodiments, a library is a soluble scFv antibody library.

The molecules of a library can be produced in vitro or in vivo, for example by expression of a molecule in vivo. Also, the molecules of a library can be displayed on any relevant support, for example, on bacterial pili (Lu et al., 1995) or on phage (Smith, 1985).

A library can comprise a random collection of molecules. Alternatively, a library can comprise a collection of molecules having a bias for a particular sequence, structure, conformation, or in the case of an antibody library, can be biased in favor of antibodies that bind to a particular antigen or antigens (for example, an antigen present on or in an HCV virion and/or encoded by an HCV genome). See e.g., U.S. Pat. Nos. 5,264,563 and 5,824,483. Methods for preparing libraries containing diverse populations of various types of molecules are known in the art, for example as described in U.S. patents cited herein above. Numerous libraries are also commercially available.

In some embodiments, a peptide library comprises peptides comprising three or more amino acids, in some embodiments at least five, six, seven, or eight amino acids, in some embodiments up to 50 amino acids or 100 amino acids, and in some embodiments up to about 200 to 300 amino acids.

The peptides can be linear, branched, or cyclic, and can include non-peptidyl moieties. The peptides can comprise naturally occurring amino acids, synthetic amino acids, genetically encoded amino acids, non-genetically encoded amino acids, and combinations thereof.

A biased peptide library can also be used, a biased library comprising peptides wherein one or more (but not all) residues of the peptides are constant. For example, an internal residue can be constant, so that the peptide sequence is represented as:

(XAA₁)ₘ-(AA)₁-(XAA₂)ₙ

where XAA₁ and XAA₂ are any amino acid, or any amino acid except cysteine, wherein XAA₁ and XAA₂ are the same or different amino acids, m and n indicate a number XAA residues, wherein m and n are independently chosen from the range of 2 residues to 20 residues in some embodiments, and from the range of 4 residues to 9 residues in some embodiments, and AA is the same amino acid for all peptides in the library. In some embodiments, AA is located at or near the center of the peptide. More specifically, in some embodiments m and n are not different by more than 2 residues; in some embodiments m and n are equal.

In some embodiments, a library is employed in which AA is tryptophan, proline, or tyrosine. In some embodiments, AA is phenylalanine, histidine, arginine, aspartate, leucine, or isoleucine. In some embodiments, AA is asparagine, serine, alanine, or methionine. In some embodiments, AA is cysteine or glycine.

In some embodiments of the presently disclosed subject matter, the library is a phage peptide library. Phage display is a method to discover peptide ligands while minimizing and optimizing the structure and function of proteins. Phage are used as a scaffold to display recombinant libraries of peptides and provide a vehicle to recover and amplify the peptides that bind to target biomolecules in vivo and/or in vitro.

The T7 phage has an icosahedral capsid made of 415 proteins encoded by gene 10 during its lytic phase. The T7 phage display system has the capacity to display peptides up to 15 amino acids in size at a high copy number (415 per phage). Unlike filamentous phage display systems, peptides displayed on the surface of T7 phage are not capable of peptide secretion. T7 phage also replicate more rapidly and are extremely robust when compared to other phage. The stability allows for bioscreening selection procedures that require persistent phage infectivity. Accordingly, the use of T7-based phage display is an aspect of some embodiments of the presently disclosed subject matter.

A phage peptide library to be used in accordance with the screening methods of the presently disclosed subject matter can also be constructed in a filamentous phage, for example M13 or an M13-derived phage. In some embodiments, the encoded antibodies are displayed at the exterior surface of the phage, for example by fusion to the product of M13 gene III. Methods for preparing M13 libraries can be found in Sambrook & Russell, 2001, among other places.

In some embodiments, a ligand that binds to a biomolecule indicative of HCV infection is an antibody or a fragment or derivative thereof. To identify antibodies, fragments, and derivatives thereof that bind to biomolecules indicative of HCV infection, libraries can be screened using the methods disclosed herein. Libraries that can be screened using the disclosed methods include, but are not limited to libraries of phage-displayed antibodies and antibody fragments, and libraries of soluble antibodies and antibody fragments.

“Fv” is the minimum antibody fragment that contains a complete antigen recognition and binding site. In a two-chain Fv species, this region consists of a dimer of one heavy and one light chain variable domain in tight, non-covalent association. In a single-chain Fv species (scFv), one heavy and one light chain variable domain can be covalently linked by a flexible peptide linker such that the light and heavy chains can associate in a “dimeric” structure analogous to that in a two-chain Fv species. It is in this configuration that the three complementarity-determining regions (CDRs) of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. For a review of scFv, see Pluckthun, 1994.

The phrase “antibodies, fragments, and derivatives thereof”, and grammatical variations thereof, refers to immunoglobulin molecules and immunologically active portions of immunoglobulin molecules; i.e., molecules that contain an antigen-binding site that specifically bind an antigen. As such, the term refers to immunoglobulin proteins, or functional portions thereof, including polyclonal antibodies, monoclonal antibodies, chimeric antibodies, hybrid antibodies, single chain antibodies (e.g., a single chain antibody represented in a phage library), mutagenized antibodies, humanized antibodies, and antibody fragments that comprise an antigen binding site (e.g., Fab and Fv antibody fragments). Thus, “antibodies, fragments, and derivatives thereof” include, but are not limited to monoclonal, chimeric, recombinant, synthetic, semi-synthetic, or chemically modified intact antibodies having for example Fv, Fab, scFv, or F(ab)₂ fragments.

The immunoglobulin molecules of the presently disclosed subject matter can be of any type (e.g., IgG, IgE, IgM, IgD, IgA and IgY), class (e.g., IgG₁, IgG₂, IgG₃, IgG₄, IgA₁, and IgA₂), or subclass of immunoglobulin molecule. In some embodiments, the antibodies are human antigen-binding antibody fragments of the presently disclosed subject matter and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding antibody fragments, including single-chain antibodies, can comprise the variable region(s) alone or in combination with the entirety or a portion of the following: hinge region, CH1, CH2, and CH3 domains.

The antibodies, fragments, and derivatives thereof of the presently disclosed subject matter can be from any animal origin including birds and mammals. For example, the antibodies can be human, murine (e.g., mouse and rat), donkey, sheep, rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described, for example, in U.S. Pat. No. 5,939,598.

In some embodiments of the presently disclosed subject matter, a peptide library and/or an antibody library (for example, a library of scFv antibodies) can be used to perform the disclosed screening methods. Such a library can be constructed, for example, in M13 or an M13-derived phage. See e.g., U.S. Pat. Nos. 6,593,081; 6,225,447; 5,580,717; and 5,702,892, all incorporated by reference herein.

Phage-displayed recombinant peptides and/or antibodies are genetically cloned and expressed on the tip of the M13 bacteriophage (McCafferty et al., 1990). M13 phage infects E. coli that carry an F′ episome (plasmid) and constantly produce and secrete intact M13 virus particles without lysing the host cell. The components of the M13 phage include phage DNA, coat proteins, gene III attachment proteins, and other proteins that are fused to the phage proteins. There are 3-5 copies of the gene III attachment proteins located on the exterior of the phage that are responsible for phage attachment to receptors on E. coli cells.

In some embodiments, M13 phage-displayed recombinant antibodies can be created by linking DNA from antibody-producing B lymphocytes to the phage gene III DNA using the pCANTAB vector (Amersham Biosciences, Piscataway, N.J., United States of America). The proteins encoded by the antibody in gene III DNA are fused to one another to produce an antibody-gene III fusion protein. A bacteriophage carrying the gene fusion will simultaneously contain the antibody DNA and express an antibody molecule on the gene III protein.

A representative, non-limiting approach to obtain and characterize antigen-specific recombinant antibodies or antibody fragments (for example, scFv antibodies or human Fab antibodies) is as follows. Phage antibody selections can be performed using antigens immobilized on solid supports or biotinylated antigens and streptavidin magnetic beads. An aliquot of a phage antibody library can be applied to the antigen. Nonspecific phage antibodies are thereafter washed off of the antigen, and phage that encode bound antibodies can be eluted and used to infect E. coli. Infected E. coli can be plated and rescued with helper phage to produce an antigen-enriched phage antibody library. The antigen-enriched library (i.e., a library pre-selected for binding to a particular antigen of interest) can be used in a second round of selection for binding to the antigen. Subsequent rounds of selection on antigen and helper phage rescue can be used until the desired antigen-specific antibodies are obtained. Colonies stemming for phage antibody selections can be picked from agar plates manually or by using a colony picker (for example, the QPix2 Colony Picker from Genetix USA Inc., Boston, Mass., United States of America). Picked colonies can then transferred to appropriate vessels, for example microwell plates, and can be used to produce soluble recombinant antibodies.

Phage-displayed recombinant antibodies have several advantages over polyclonal antibodies or hybridoma-derived monoclonal antibodies. Phage-displayed antibodies can be generated within 8 days. Recombinant antibody clones can be easily selected by panning a population of phage-displayed antibodies against immobilized antigen (McCafferty et al., 1990). The antibody protein and antibody DNA are simultaneously contained in one phage particle (Better et al. (1988) Science 240:1041-3). Liters of phage-displayed recombinant antibodies can be produced inexpensively from bacterial culture supernatant and the phage antibodies can be used directly in immunoassays without purification. Phage display technology makes possible the direct isolation of monovalent scFv antibodies. The small size of scFv antibodies makes it the antibody format of choice for penetration of cells and/or tissues and for rapid clearance from the blood (Adams, 1998; Adams et al., 1995; Yokota et al., 1992). The human phage antibody library can be used to develop antibodies suitable for clinical trials. Human scFv antibodies have entered clinical trials (Hoogenboom & Winter, 1992). Anti-melanoma antibodies have been developed using these phage libraries (Cai & Garen, 1995), as well as antibodies to antigens found in ovarian carcinoma (Figini et al. (1998) Cancer Res 58:991-996).

The recombinant phage can comprise antibody encoding nucleic acids isolated from any suitable vertebrate species, including in some embodiments mammalian species such as human, mouse, and rat. Thus, in some embodiments the recombinant phage encode an antibody wherein both the variable and constant regions are encoded by nucleic acids isolated from the same species (for example, human, mouse, or rat). In some embodiments, the recombinant phage encode chimeric antibodies, wherein the phrase “chimeric antibodies” (and grammatical variations thereof) refers to antibodies having variable and constant domain regions that are derived from different species. For example, in some embodiments the chimeric antibodies are antibodies having murine variable domains and human constant domains.

The scFv antibodies of the presently disclosed subject matter also include humanized scFv antibodies. Humanized forms of non-human (for example, murine) scFv antibodies are chimeric scFv antibodies that contain minimal sequence derived from non-human immunoglobulins. Humanized scFv antibodies include human scFvs in which residues from a complementarity-determining region (CDR) are encoded by a nucleic acid encoding a CDR of a non-human species such as mouse, having the desired specificity, affinity, and capacity. In some instances, Fv framework residues of the human immunoglobulin are replaced by corresponding non-human residues. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; Presta, 1992). Thus, as used herein, the term “humanized” encompasses chimeric antibodies comprising a human constant region, including those antibodies wherein all of the residues are encoded by a human nucleic acid (see e.g., Shalaby et al., 1992; Mocikat et al., 1994).

The presently disclosed subject matter also provides molecules identified by the screening methods disclosed herein.

IV.B. In Vitro Screening Reagents

In order to test the identified candidate molecules for an ability to modulate lipid metabolism, an in vitro system can be established that comprises a plurality of cells (e.g., liver cells) that express an HCV Core polypeptide with various amino acids in the positions corresponding to amino acids 182 and 186 of SEQ ID NO: 2. In some embodiments, the plurality of cells comprise cells isolated from a subject that has been infected with an HCV isolate of the desired genotype.

In some embodiments, an in vitro system can be developed that comprises cells that stably express various Core polypeptides and that includes cells that are immortalized, thereby eliminating the need to constantly re-isolate cells of the desired type. To that end, the presently disclosed subject matter also provides a cell transformed with a nucleic acid molecule encoding an HCV Core polypeptide having desired amino acids in the positions corresponding to amino acids 182 and 186 of SEQ ID NO: 2. The transformed cells can be any cell type, and in some embodiments are liver cells. Representative liver cells include, but are not limited to Huh-7 cells and derivatives thereof (see e.g., Nakabayashi et al., 1982; Blight et al., 2003), HepG2 and Hep3B cells (available from the American Type Culture Collection, Manassas, Va., United States of America), and 5H cells (gift of Dr. Markos Rojkind, The George Washington University Medical Center, Washington D.C., United States of America).

To produce a cell line that stably expresses an HCV Core polypeptide with a desired sequence, a recombinant nucleic acid that encodes an HCV Core polypeptide can be produced using techniques that are known in the art. Exemplary Core polypeptides include those that have various pairs of amino acids at positions that correspond to amino acids 182 and 186 of SEQ ID NO: 2, including particularly HCV Core polypeptides that have the FV or LI pairs that have been associated with steatosis. Additional recombinant nucleic acids that encode other pairs at these positions (e.g., FI and LV) can also be produced. Standard molecular biology techniques can then be employed to stably express the recombinant Core polypeptides in cells (see e.g., Sambrook & Russell, 2001 for procedures that can be employed for generating recombinant nucleic acids and transforming the same into various cell types).

Alternatively, HCV particles can be isolated from subjects infected with appropriate HCV genotypes and these can be used to infect cells lines in vitro. While HCV replication in in vitro infected cells has proved technically challenging, several successful in vitro infection studies have recently been described. See Zhong et al., 2005; Wakita et al., 2005; Lindenbach et al., 2005; and Pietschmann et al., 2006, each of which is in incorporated herein by reference in its entirety.

### V. Methods for Modulating Lipid Accumulation

The presently disclosed subject matter also provides methods for modulating lipid accumulation in a cell in a subject in need thereof. In some embodiments, the methods comprise administering to a subject in need thereof a therapeutically effective amount of a composition identified by the screening methods disclosed herein. In some embodiments, the lipid accumulation is associated with steatosis, and in some embodiments the steatosis comprises lipid accumulation in the liver of the subject. In some embodiments, the steatosis is incident to infection with HCV.

V.A. Subjects

The subjects treated using, or in whom a tendency to develop steatosis is predicted by, the presently disclosed subject matter in its many embodiments is desirably a human subject, although it is to be understood that the principles of the presently disclosed subject matter indicate that the presently disclosed subject matter is effective with respect to all vertebrate animals, including mammals, which are intended to be included in the term “subject”. Moreover, a mammal is understood to include any mammalian species in which treatment or prevention of a disease is desirable, particularly agricultural and domestic mammalian species. For example, the presently disclosed subject matter is applicable to the treatment of livestock.

The methods of the presently disclosed subject matter are particularly useful in the treatment of warm-blooded vertebrates. Thus, the presently disclosed subject matter concerns mammals and birds.

More particularly provided is the treatment of mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economic importance to humans. Thus, provided is the treatment of livestock, including, but not limited to, domesticated swine (pigs and hogs), ruminants, horses, poultry, and the like.

V.B. Formulations

The compositions of the presently disclosed subject matter and other reagents employed in the methods of the presently disclosed subject matter comprise in some embodiments a composition that includes a carrier, particularly a pharmaceutically acceptable carrier or diluent. Any suitable pharmaceutical formulation can be used to prepare the compositions for administration to a subject.

For example, suitable formulations can include aqueous and non-aqueous sterile injection solutions that can contain anti-oxidants, buffers, bacteriostatics, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are SDS, in one example in the range of 0.1 to 10 mg/ml, in another example about 2.0 mg/ml; and/or mannitol or another sugar, for example in the range of 10 to 100 mg/ml, in another example about 30 mg/ml; and/or phosphate-buffered saline (PBS).

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The therapeutic regimens and compositions of the presently disclosed subject matter can be used with additional adjuvants or biological response modifiers including, but not limited to, cytokines and other immunomodulating compounds.

V.C. Administration

Administration of the compositions of the presently disclosed subject matter can be by any method known to one of ordinary skill in the art, including, but not limited to intravenous administration, intrasynovial administration, transdermal administration, intramuscular administration, subcutaneous administration, topical administration, rectal administration, intravaginal administration, oral administration, buccal administration, nasal administration, parenteral administration, inhalation, insufflation, and direct administration to a cell or tissue of interest. In some embodiments, suitable methods for administration of a composition of the presently disclosed subject matter include but are not limited to intravenous injection. The particular mode of administering a composition of the presently disclosed subject matter depends on various factors, including the distribution and abundance of cells to be treated, the compound employed, additional tissue- or cell-targeting features of the compound, and mechanisms for metabolism or removal of the compound from its site of administration.

V.D. Dose

An effective dose of a composition of the presently disclosed subject matter is administered to a subject in need thereof. An “effective amount” or a “therapeutic amount” is an amount of a therapeutic composition sufficient to produce a measurable response (e.g., a biologically or clinically relevant response in a subject being treated). Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the therapeutic composition, the route of administration, combination with other drugs or treatments, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved. The potency of a composition can vary, and therefore a “treatment effective amount” can vary. However, using the assay methods described herein, one skilled in the art can readily assess the potency and efficacy of a candidate compound of the presently disclosed subject matter and adjust the therapeutic regimen accordingly.

After review of the disclosure of the presently disclosed subject matter presented herein, one of ordinary skill in the art can tailor the dosages to an individual subject, taking into account the particular formulation, method of administration to be used with the composition, and particular disease treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

### EXAMPLES

The following Examples have been included to illustrate modes of the presently disclosed subject matter. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### Subject Selection

Eight highly pedigreed subject samples were selected from a large biorepository in order to perform initial studies and control for other factors that might contribute to steatosis. The repository contained pertinent demographic, biochemical, virologic and histologic data. Matched serum samples from over 1000 subjects with chronic HCV infection were also available. The study protocol was reviewed and approved by the Duke University Institutional Review Board (Durham, N.C., United States of America.

Selection criteria included treatment naïve subjects with HCV genotype 3a infection with complete data and available serum samples. As these subjects were enrolled in clinical treatment protocols, abstinence from alcohol was required for 12 months prior to therapy. Liver biopsies from a subset of 26 subjects were examined and subjects were divided into those with evidence of histologic steatosis [grade 2 (30-59% cells with fat) and grade 3 (greater than or equal to 60% cells with fat)] on liver biopsy and those without any evidence of steatosis [grade 0 (0-2% cells with fat)] as judged by an experienced pathologist blinded to subject information. Four samples were selected from each group for this initial study.

Table 1 summarizes the relevant demographic data. There were no significant differences in baseline demographic variables between subjects with and without steatosis (see Table 1). There were statistically significant differences in steatosis grade, with the steatosis group having more severe disease compared to the non-steatosis group (p<0.001). Subjects without steatosis had more advanced fibrosis.

TABLE 1
Summary of Subject Data

| | Steatosis | Non-Steatosis | |
|---|---|---|---|
| Characteristics | group | group | p values |
| Number | 4 | 4 | nd |
| % white | 100% | 75% | nd |
| % Males | 75% | 75% | nd |
| Age (±SD years) | 43.5 ± 2.38 | 38 ± 5.89 | 0.60 |
| Body Mass Index | 26.825 ± 3.47 | 24.825 ± 3.82 | 0.87 |
| (±SD) | | | |
| % with Diabetes | 0% (1)* | 0% | nd |
| diagnosed | | | |
| ALT | 95.75 ± 30.3 | 119 ± 78.7 | 0.15 |
| Total Cholesterol | 162.25 | 168.5 (2)* | nd |
| (±SD mg/dl) | | | |
| Triglycerides | 111.75 | 130 (2)* | nd |
| (±SD mg/dl) | | | |
| HCV viral load | 8 | 2.61 | 0.1 |
| (±SD × 10⁶) | | | |
| copies/ml) | | | |
| Steatosis grade | 3 ± 0 | 0.25 ± 0.5 | <0.001 (95% |
| (average ± SD) | | | CI: 1.95-3.54) |
| Metavir Fibrosis | 1 ± 0 | 1.75 ± 1.5 | <0.001 (95% |
| score | | | CI: −1.63-3.13) |
| (average ± SD) | | | |

Key: Steatosis grade: grade 0 (0-2% hepatocytes containing fat), grade 1 (3-29%), grade 2 (30-59%) and grade 3 (≧60%); nd: not done
*the number in parenthesis corresponds to the number of subjects with no data for that category

### Example 2

### Viral RNA Isolation and RT-PCR

Serum samples stored at −80° C. and viral RNA was isolated from thawed 200 μl aliquots using the QIAAMP® MINELUTE™ Virus Spin kit (Qiagen Inc., Valencia, Calif., United States of America) according to the manufacturer's protocol. RT-PCR was performed using the SUPERSCRIPT™ III One-Step RT-PCR system (INVITROGEN™ Corp., Carlsbad, Calif., United States of America) according to the manufacturer's recommended protocol. Custom primers were designed using sequence information available in the HCV Sequence Database (Los Alamos National Laboratory, Los Alamos, N. Mex., United States of America) to amplify the Core region from genotype 3 isolates (see Table 2).

TABLE 2
Custom Oligonucleotide Primers Sequences

| | |
|---|---|
| HCV Core Genotype 3 forward: | ATGCGAATTCGCCACCATGAGCACAC |
| | TTCCTAAA (SEQ ID NO: 3) |
| HCV Core Genotype 3 reverse: | AGTCTCTAGATCATCAACTTGCTGCT |
| | GGATG (SEQ ID NO: 4) |
| HCV Core mutant V186I reverse*: | AGTCTCTAGATCATCAACTTGCTGCT |
| | GGATGAATTAAGCAAGA |
| | (SEQ ID NO: 5) |
| HCV Core mutant I186V reverse*: | AGTCTCTAGATCATCAACTTGCTGCT |
| | GGATGAACTAAGCAAGA |
| | (SEQ ID NO: 6) |
| HCV Core mutant L182F reverse*: | AGTCTCTAGATCATCAACTTGCTGCT |
| | GGATGAATTAAGCAAGAGAACAGAGC |
| | TAGAAG (SEQ ID NO: 7) |

*Codons for introducing site-specific mutations (see Example 4) are in bold

100-200 ng of viral RNA was used per reaction, and the reactions conditions were as follows:

| Step | Conditions |
|---|---|
| Reverse Transcription | 50° C. for 30 minutes |
| Denaturation | 94° C. for 2 minutes |
| Amplification (45 cycles) | |
| Denaturation; | 94° C. for 30 seconds; |
| Annealing; and | 64° C. for 30 seconds; |
| Extension | 68° C. for 45 seconds |
| Final Extension | 68° C. for 5 minutes |

Amplicons were analyzed by agarose gel electrophoresis to confirm appropriate sizes for the amplified fragments (see FIG. 1B).

### Example 3

### Cloning and Sequence Analysis

Products obtained from RT-PCR above were then purified using a QIAQUICK® PCR purification kit (Qiagen Inc., Valencia, Calif., United States of America), digested in the restriction enzymes Eco RI and Xba I and then purified again. The digested insert was then ligated into a pre-digested vector, pAC-CMV, using T4 DNA ligase (New England Biolabs, Ipswich, Mass., United States of America). 5 μl of the ligation reaction was transformed into competent E. coli cells and grown overnight at 37° C. on LB media containing ampicillin. Colonies were grown overnight with ampicillin selection and plasmid DNA was isolated using QIAAMP® Spin Miniprep Kit (Qiagen Inc., Valencia, Calif., United States of America). Plasmids containing the HCV Core insert were screened by restriction endonuclease digestion and then sequenced.

Sequence analysis was performed by the Duke University Medical Center DNA Sequence Analysis core facility using primers complementary to the CMV promoter (pCMV forward: 5′-CGCAAATGGGCGGTAGGCGTG-3′; SEQ ID NO: 8) and SV40 poly A sequence (SV40 reverse primer: 5′-TCTCTGTAGGTAGTTTGTCC-3′; SEQ ID NO: 9) for the 5′ and 3′ directions respectively. The primers were READYMADE™ Primers from Integrated DNA Technologies (Coralville, Iowa, United States of America). Each isolate of viral RNA was amplified, cloned, and sequenced in triplicate to minimize any polymerase introduced errors. Nucleotide sequence results and predicted amino acid sequences were compared using the following programs: Blast, Blast 2 (NCBI), Clustal, and Transeq (EMBL-EBI).

Comparisons were made among the predicted amino acid sequences for all 8 samples. No one single amino acid substitution segregated the Core isolates into those patients with steatosis and without steatosis, but detailed analysis within the domain 3 region of Core yielded important differences at residues 182 and 186. As illustrated in FIG. 1A, all Core isolates from patients with steatosis had the amino acid pair phenylalanine-valine (FV) or leucine-isoleucine (LI) at amino acids 182-186. The Core isolates from patients without steatosis had the pair phenylalanine-isoleucine (FI) at these locations. There was only one patient sample in the non-steatosis group that yielded discordant sequence results. Statistical analysis showed the sequence differences to be significantly related to their respective steatosis phenotype (LI with steatosis, p=0.03; FI with no steatosis, p=0.005).

### Example 4

### Generation of Clones and Mutants, Transfection, and Western Blot Analysis

After the above sequences were analyzed, Core isolates were cloned for in vitro expression. Three of the isolates, HCV1, HCV11, and HCV12, were mutated using PCR with custom 3′ primers that were designed to change the amino acid at position 182 or 186 depending on the clone: HCV1-V1861; HCV11-1186V; HCV12-L182F (see Table 2). The resulting mutant clones were expected to have their “steatogenic” phenotype switched compared to the corresponding parent clone.

Particularly, the pAC-CMV plasmids containing HCV Core from 3 of the clones (HCV1, HCV11 and HCV 12) were reamplified by PCR using the IPROOF™ High Fidelity DNA Polymerase (Bio-Rad Laboratories, Inc., Hercules, Calif., United States of America) with either the original pair of HCV genotype 3 Core primers or with a substituted custom 3′ primer that was designed to mutate the nucleotides coding for amino acids 182 or 186 (see Table 2). These amplicons were digested with Eco RI and Xba I and ligated into the vector pcDNA3.1 V5-His A (INVITROGEN™ Corp., Carlsbad, Calif., United States of America) as described above. Plasmids were screened and sequence analysis was performed as described above. The Core sequence from clone HCV-N (genotype 1b; GENBANK Accession No. AF139594) was cloned and used as a comparator.

In vitro transfections were performed in Huh-7 cells. Briefly, the cells were passaged into 6 well plates the day prior to transfection at approximately 50% confluence. Transfection was performed using LIPOFECTAMINE™ and PLUS™ reagents in OPTI-MEM® media (INVITROGEN™ Corp., Carlsbad, Calif., United States of America) with 1 μg of plasmid per well according to the manufacturer's recommended protocol. After 72 hours cell lysates were prepared using Passive Lysis Buffer (Promega Corp., Madison, Wis., United States of America). Lysates were analyzed using SDS-PAGE electrophoresis and western blot with anti-HCV Core antibody (Austral Biologicals, San Ramon, Calif., United States of America), goat anti-mouse secondary antibody conjugated to horseradish peroxidase (Santa Cruz Biologicals, Santa Cruz, Calif., United States of America). Protein bands were visualized using a chemiluminescence kit (Amersham Biosciences, Piscataway, N.J., United States of America) and images were captured on BioMax film (Eastman Kodak Co., Rochester, N.Y., United States of America).

### Example 5

### Immunofluorescence and Oil Red O Staining and Image Analysis

HepG2 and 5H cells were transfected using an adenovirus component “piggyback” method as described in Kohout et al., 1996. Briefly, cells were passaged into 4 well chamber slides at 60-70% confluence and the HCV Core containing clones were applied after being incubated with empty adenovirus, poly-L lysine and DMEM media with 25 mM HEPES and no serum for 30 minutes successively. 300 μl of the 15 ml original volume was added to each well and incubated at 37° C./5% CO₂ for 2 hours. 1 ml of regular serum containing media was then added to each well and cells were incubated overnight. Media was replaced at 24 hours and then at 48 hours cells were washed with PBS and fixed with 4% paraformaldehyde for 30 minutes at 37° C.

Cells were permeabilized in PBS with 0.1% Triton-X for 5 minutes and then blocked in PBS with 5% BSA for 10 minutes. Cells were stained with anti-HCV Core antibody (Affinity BioReagents, Golden, Colo., United States of America) in PBS/5% BSA at a concentration of 1:250 for 30 minutes at 37° C. Cells were washed with PBS then stained with Alexa-Fluor goat anti-mouse IgG, secondary antibody (MOLECULAR PROBES™, a division of INVITROGEN™ Corp., Carlsbad, Calif., United States of America) at a concentration of 1:400 for 30 minutes at 37° C. Cells were then washed in PBS, fixed again as above for 10 minutes, and stained with DAPI in methanol at a concentration of 1:1000 for 3 minutes at room temperature. Cells were washed with PBS twice and then ORO staining was performed immediately following by first washing the cells with propylene glycol three times for 5 minutes each. ORO stain in propylene glycol (Newcomer Supply, Madison, Wis., United States of America) was applied to cells for 7 minutes and then cells were washed 85% propylene glycol for 3 minutes. Cells were then rinsed in distilled water twice and then mounted with PBS/glycerol 1:1. Slides were examined using an Axiovert 200 microscope (Carl Zeiss MicroImaging, Inc., Thornwood, N.Y., United States of America) at 63-100× magnification with epifluorescent illumination. Images were recorded using an AxioCam HRC camera (Carl Zeiss MicroImaging, Inc., Thornwood, N.Y., United States of America) that was controlled by AxioVision 4.4 software (Carl Zeiss MicroImaging, Inc., Thornwood, N.Y., United States of America). Ten 63× high power fields were examined for each clone in duplicate so that 20 fields per clone could be analyzed.

Image analysis was performed using METAMORPH® version 7 software (Molecular Devices Corp., Downingtown, Pa., United States of America). Briefly, a region was drawn around each cell or cells that were fluorescent green on the FITC image. A green color threshold was applied and data was collected on percent area thresholded, min/max intensity and average intensity. Next, the region was transferred to the same coordinates on the corresponding brightfield image. A red threshold was applied and data was collected on percent area thresholded, min/max intensity and average intensity. Results were then analyzed to calculate average percent area thresholded red on the sets of images for each HCV clone as well as standard deviation and standard error of the mean.

A protocol that combined immunofluorescence (IF) staining for HCV Core polypeptide and Oil Red O (ORO) histologic staining was then designed to address the following questions: 1) whether in vitro expression of clones derived from patients with steatosis resulted in increased intracellular lipid accumulation within cells; and 2) whether this lipid accumulation was significantly higher than when the non-steatosis clones were expressed.

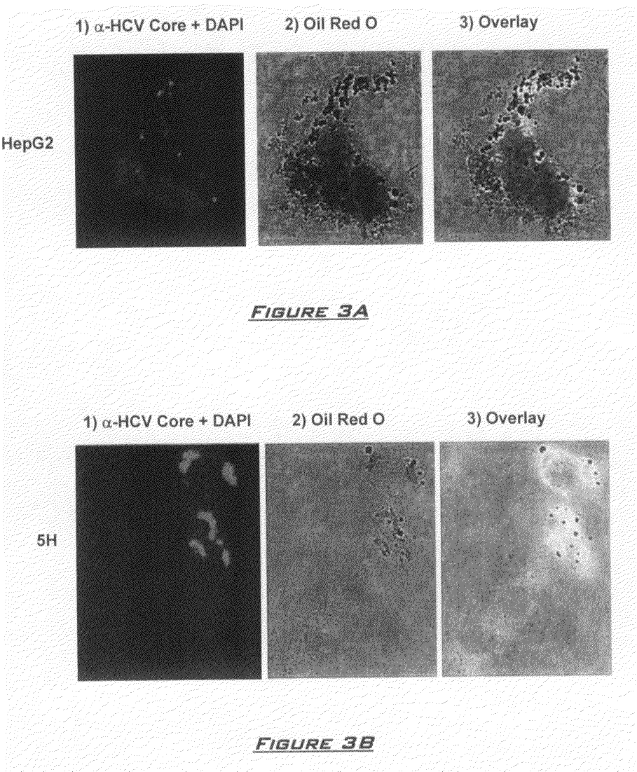

FIG. 3A shows HepG2 cells that were transfected with HCV Core and then analyzed at 48 hours with IF and ORO staining. Although there are many lipid droplets within the cell staining positive for HCV Core, several other cells also appeared to have significant intracellular lipid. This “background” steatosis was seen in both Huh-7 and Hep3B cell lines, so analysis of relatively subtle differences between the steatosis and non-steatosis clones would have been unavailing.

Transfection of HCV Core was repeated in 5H cells, a clonally derived rat liver cell line with epithelial and stellate cell characteristics that has previously been used for lipid metabolism experiments (see e.g., Greenwel et al., 1993; Kannangai et al., 2005). This cell line has little or no lipid present under normal culture conditions.

Preliminary studies showed equivalent levels of protein expression as detected by IF between HCV Core clones. FIG. 3B shows 5H cells transfected with HCV Core polypeptide and analyzed after 48 hours by IF and ORO. Cells expressing HCV Core polypeptide contained numerous lipid droplets, while cells not expressing HCV Core or a control protein (green fluorescent protein; GFP) had minimal detectable intracellular lipid in the field evaluated.

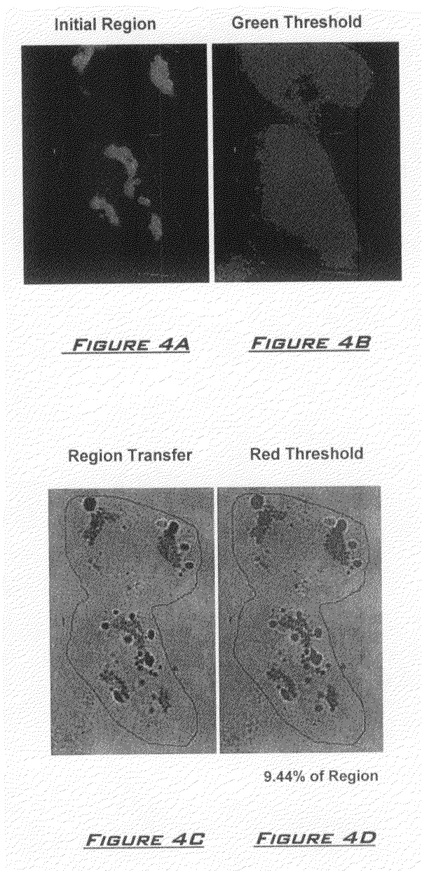

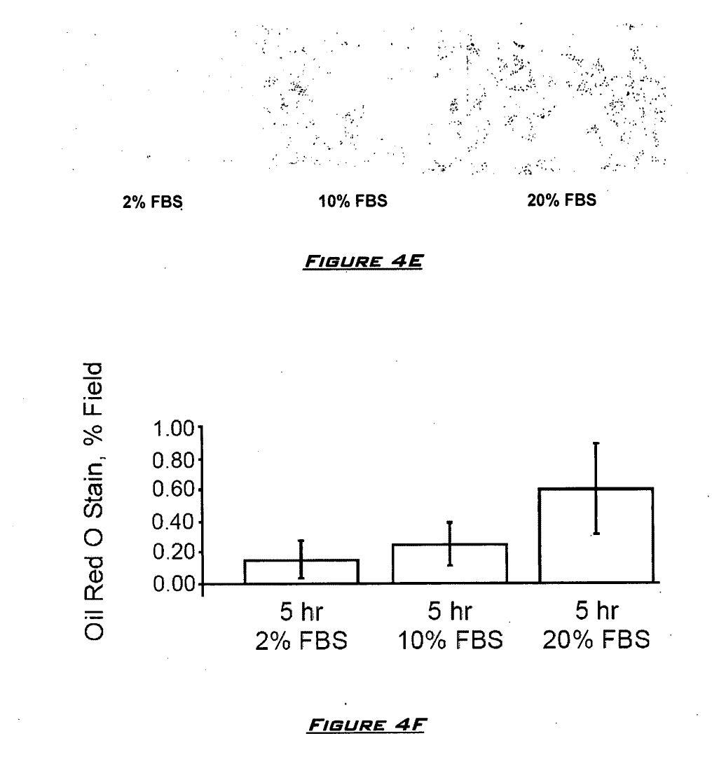

A method for objective quantitation was developed to analyze lipid accumulation within transfected cells. Detecting subtle differences using conventional biochemical techniques would have been difficult as the overall number of transfected cells per sample was quite low (5%). Image analysis was used to obtain quantitative measures of lipid accumulation (see FIG. 4). Regions were set on the IF image and directly transferred to the ORO image and total red was analyzed and recorded. An advantage of this method was that no subjective bias from an observer was introduced at this stage.

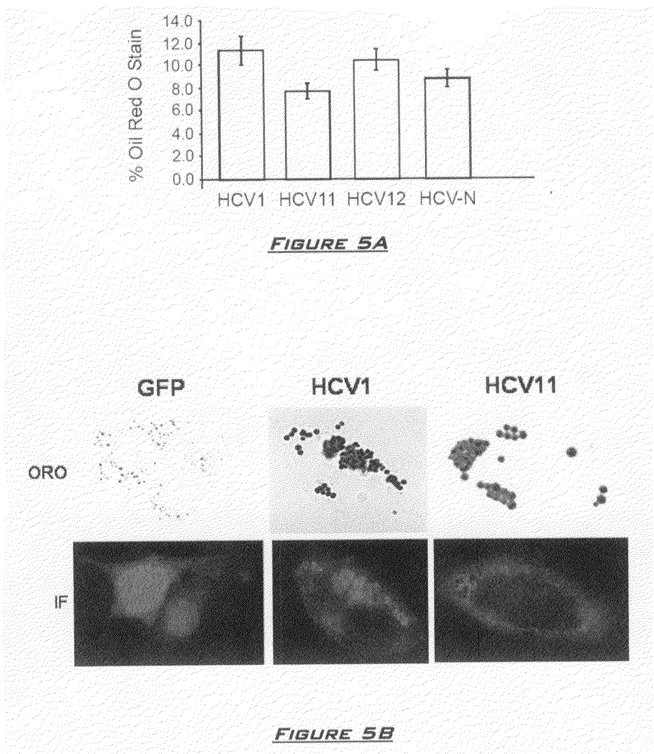

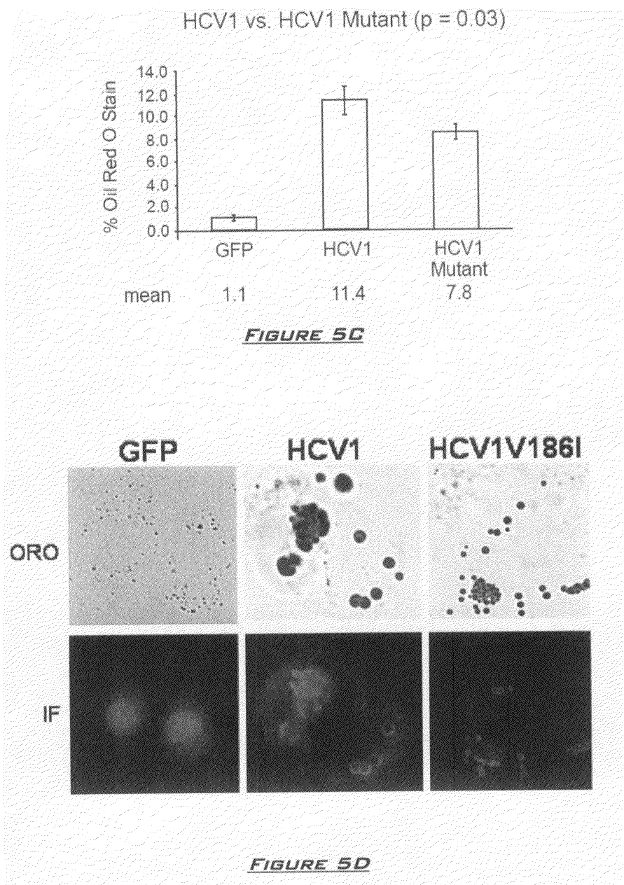

Transfections were performed in 5H cells using each of the 3 HCV Core clones expressed along with their corresponding mutants. That the appropriate protein was being translated in each case was confirmed by Western blotting, a representative example of which is shown in FIG. 2. GFP and an empty vector were used as controls. Transfections were performed in duplicate, followed by IF and ORO staining after 48 hours. Image analysis was performed on twenty high power field images for each transfected well on the chamber slide (see FIG. 5). Expression of a control (GFP) protein resulted in minimal (1%) ORO stain in analyzed regions while expression of all of the HCV Core wild-type and mutant clones caused increased lipid accumulation. The distribution of these lipid droplets was primarily perinuclear, except in cells with lipid overload in which case the droplets were distributed throughout the cytoplasm.

There were important quantitative differences between the clones compared to each other and their respective mutants. Expression of the HCV1 clone, which was associated with steatosis, resulted in significantly more ORO stain per region analyzed compared to expression of the HCV11 clone that was not associated with steatosis (11.4%±6.7% vs. 7.8%±3.3%; p=0.02). Further analysis revealed that when the HCV1 clone was compared with its mutant clone, with the amino acid at position 186 changed from V to I that reversed its steatosis phenotype, the HCV1 mutant clone had 27% less ORO stain per region compared to the parent clone (11.4%±6.7% vs. 8.3%±4.8%; p=0.03).

Similar experiments were performed with HCV12, another steatosis clone with the LI amino acid pair, by comparing it to its mutant clone, which had the amino acid at position 182 changed from L to F. There was a 37% decrease in the amount of intracellular fat by ORO in the mutant clone compared to the HCV12 parent clone that was statistically significant (p=0.01).

### Example 6

### Statistical Analysis

Statistical comparisons between groups were made using Intercooled Stata 8.0 (StataCorp LP, College Station, Tex., United States of America). For replicate experiments, data are reported as means±SD and SEM. Comparisons between groups were performed used the Student's t-test for % area that was thresholded red. Significance was accepted at the 5% level.

### Example 7

### Construction and Analysis of GFP-HCV Core Protein Domain Fusion Polypeptides

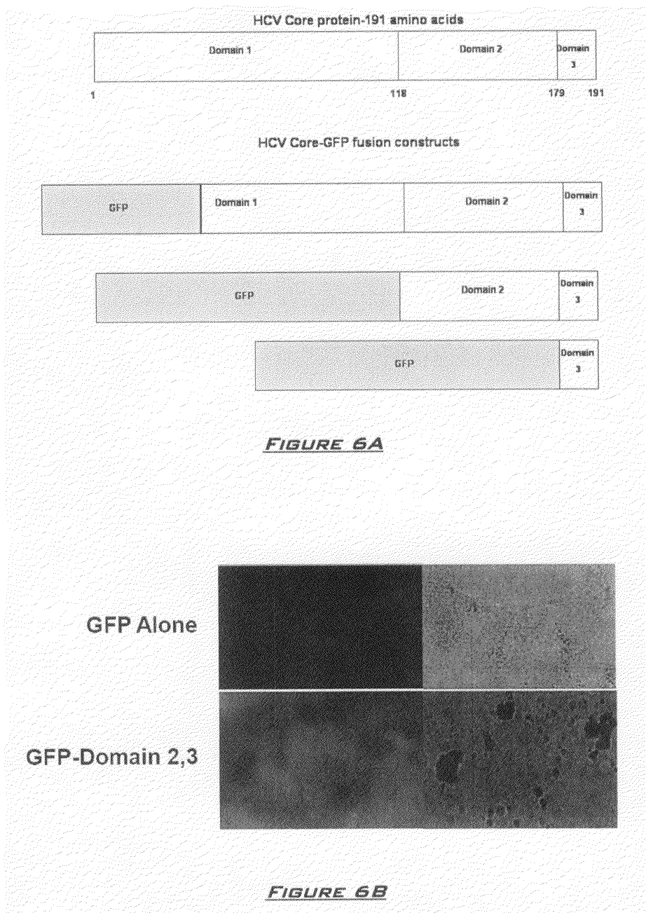

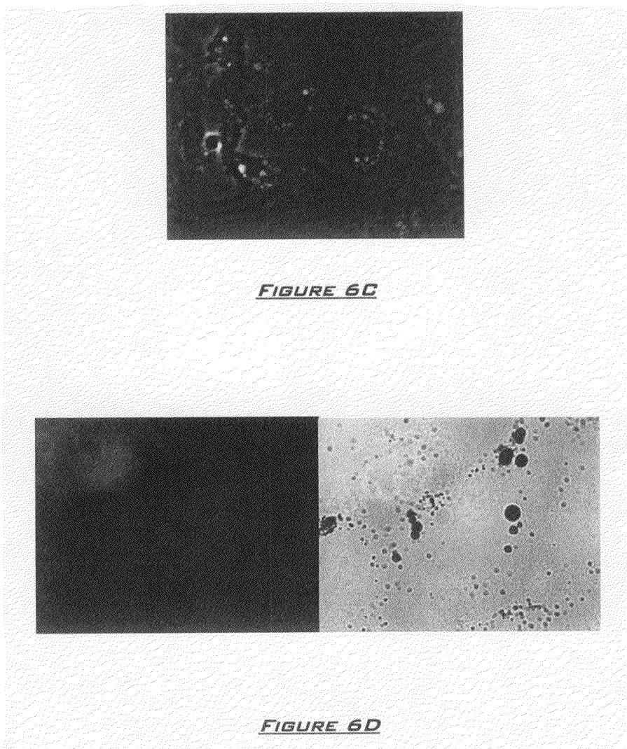

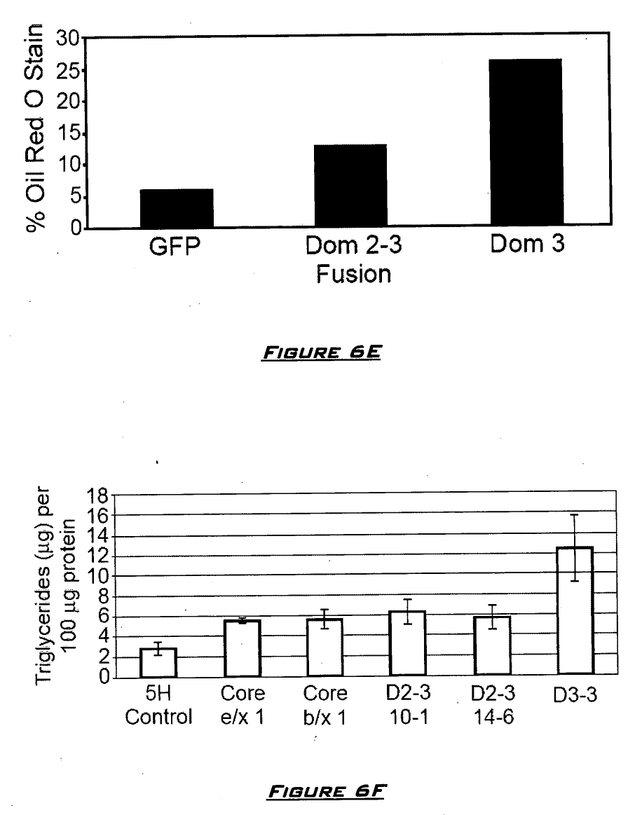

GFP fusion constructs were generated using gene specific primers cloned by restriction digest and ligation into a plasmid containing an enhanced GFP coding sequence derived from pEGFP-C1 (GENBANK® Accession No. U55763; CLONTECH Laboratories, Inc., Palo Alto, Calif., United States of America). Briefly, oligonucleotide primers were designed that allowed the full GFP coding sequence (nucleotides 613-1410 of GENBANK® Accession No. U55763) to be fused at its 3′ end to a coding sequence for HCV Core Protein Domains 1-3 (i.e., amino acids 1-191 of SEQ ID NO: 2), Domains 2 and 3 (i.e., amino acids 118-191 of SEQ ID NO: 2), or Domain 3 (i.e., amino acids 179-191 of SEQ ID NO: 2). These constructs are depicted in FIG. 6A. Sequencing of the resulting clones was employed to confirm the proper coding sequence and the maintenance of the proper reading frame. Appropriate clones were transfected into 5H cells and selected with G418 for transformants. G418 resistant colonies were picked for each clone and employed in subsequent experiments.

Staining and microscopy analyses were performed as set forth hereinabove. The results are depicted in FIGS. 6B-6E. FIG. 6B is a comparison of stable cells expressing GFP alone compared to cells expressing GFP fused to Domains 2 and 3 of HCV Core. Lipid aggregates were present in the Domain 2, 3 expressing cells. FIG. 6C is a brightfield microscope photo of cells expressing GFP fused to Domain 3 alone. Multiple, large cytoplasmic vacuoles were present in almost all cells. FIG. 6D depicts Oil Red 0 staining of cells expressing Domain 3 alone. The large vacuoles seen on the fluorescent image overlapped with the large lipid containing vacuoles in the brightfield image. FIG. 6E is a bar graph depicting the results of METAMORPH® analysis of stable cells expressing Core deletion constructs. Increased amounts of Oil Red 0 stain was observed in cells expressing GFP-Core deletion constructs. Cells expressing Domain 3 alone had the highest amount of intracellular lipid (26%), which was significantly higher than cells expressing Domains 2 and 3 or GFP alone (p<0.00001 for both). Cells expressing Domains 2 and 3 also had significantly more lipid than cells with GFP alone (p=0.002).

The triglyceride contents of transformed cells expressing Core deletion constructs were also analyzed using the Serum Triglyceride Determination Kit (Catalogue No. TR0100; Sigma-Aldrich Co., St. Louis, Mo., United States of America) according to the manufacturer's instructions. Increased amounts of triglycerides per 100 μg total protein in cells expressing GFP-Core deletion constructs was observed (see FIG. 6F). Cells expressing Domain 3 alone had the highest amount of triglycerides (12.4%), which was significantly higher than cells expressing Domains 2 and 3 or 5H control cells (p=0.01 for both). Cells expressing Domains 2 and 3 had significantly more lipid than control cells (p=0.02).

### Discussion of Examples 1-7

As disclosed herein, particular amino acid pairs at positions 182 and 186 of the HCV Core polypeptide correlated with the presence of intrahepatic steatosis in a small group of carefully selected patients infected with HCV genotype 3a. These sequence differences segregated patients with and without steatosis in all cases except for one. In vitro expression of these clones led to increased intracellular fat as detected by Oil Red staining. Significant differences in the amount of intracellular lipid when steatosis associated clones were expressed compared to non-steatosis clones was also observed.

Reversal of the steatosis phenotype through induced mutations at positions 182/186 significantly decreased the amount of intracellular lipid in transfected cells. These findings suggested that this region of the Core polypeptide plays a role in the regulation of cellular lipid metabolism or trafficking.

Previous reports also showed there are regions within domain 2 that are common to many of the genotypes that determine lipid droplet association. After reviewing reference sequences, these amino acid polymorphisms within domain 3 appear to be specific to certain genotype 3 isolates.

Domain 3 of HCV Core polypeptide is the E1 signal peptide region that facilitates cleavage of Core to the mature form of the protein and allows for proper cleavage at the Core-E1 junction by host signal peptidases. The fate of the domain 3 peptide after both cleavage events is unknown. While applicants do not wish to be bound by any particular theory of operation, given the presently disclosed findings it is possible that the role of domain 3 differs between genotypes and might involve interactions with host proteins within the ER membrane that mediate lipid metabolism and trafficking. This domain is predicted to form a helix, and some of the amino acids may be acting as “helix-benders”. Given this information, it is also possible that the different pairs of amino acids might alter the helix structure enough between the clones to change the nature of the interactions with host proteins.

Although the results of tests of only 8 patient samples are disclosed herein, the sequence results segregated the 2 groups of patients with statistical significance. The in vitro studies were also reproducible and support these findings. In vitro expression of these clones, and their corresponding mutants, confirmed that these amino acid differences strongly influenced the steatosis phenotype of these isolates.

### REFERENCES

All references listed hereinbelow and/or cited in the specification, including but not limited to U.S. and foreign patents and patent application publications, scientific journal articles, and database entries (e.g., GENBANK® Accession Nos., including all annotations presented therein), are incorporated herein by reference to the extent that they supplement, explain, provide a background for or teach methodology, techniques, and/or compositions employed herein.
Adams (1998) In Vivo 12:11-21.
Adams et al. (1995) Cancer Immunol Immunother 40:299-306.
Blight et al. (2003) J Virol 77:3181-3190.
Cai & Garen (1995) Proc Natl Acad Sci USA 92:6537-6541.
Figini et al. (1998) Cancer Res 58:991-996.
Forms & Bukh (1999) Clin Liver Dis 3:693-716.
GENBANK® Accession Nos. BAA04609 and D 17763.
Greenwel et al. (1993) Lab Invest 69:210-216.
Harlow & Lane (1988) Antibodies: A Laboratory Manual, Cold Spring Harbor Laboratory, Cold Spring Harbor, N.Y., United States of America.
Hezode et al. (2004) J Viral Hepatitis 11:455-458.
Hoogenboom & Winter (1992) J Mol Biol 227:381-388.
Jones et al. (1986) Nature 321:522-525.
Kannangai et al. (2005) Hum Pathol 36:341-347.
Kohout et al. (1996) Circ Res 78:971-977.
Lindenbach et al. (2005) Science 309:623-626.
Lonardo et al. (2004) Gastroenterol 126:586-597.
Lu et al. (1995) Biotechnology (NY) 13:366-372.
McCafferty et al. (1990) Nature 348:552-524.
Mocikat et al. (1994) Transplantation 57:405-411.
Moriya et al. (1997) J Gen Virol 78(Pt. 7):1527-1531.
Nakabayashi et al. (1982) Cancer Res 42:3858-3863.
Patton et al. (2004) J Hepatol 40:484-490.
Perlemuter et al. (2002) FASEB J 16:185-194.
Pietschmann et al. (2006) Proc Natl Acad Sci USA 103:7408-7413.
Pluckthun (1994) in The Pharmacology of Monoclonal Antibodies, vol. 113, pp. 269-315, Rosenburg & Moore (eds.), Springer-Verlag, New York, United States of America.
Poynard et al. (2003) Hepatol 38:75-85.
Presta (1992) Curr Op Struct Biol 2:593-596.
Ramalho (2003) Antiviral Res 60:125-127.
Regev & Schiff (2000) Clin Liver Dis 4:47-71.
Riechmann et al. (1988) Nature 332:323-329.
Romero-Gomez et al. (2003) Am J Gastroenterol 98:1135-1141.
Sambrook & Russell (2001) Molecular Cloning: A Laboratory Manual, 3rd ed. Cold Spring Harbor Laboratory Press, Cold Spring Harbor, N.Y., United States of America.
Shalaby et al. (1992) J Exp Med 175:217-225.
Shi et al. (2002) Virol 292:198-210.
Smith (1985) Science 228:1315-1317.
U.S. Pat. Nos. 5,223,409; 5,264,563; 5,498,538; 5,580,717; 5,650,489; 5,667,988; 5,702,892; 5,738,996; 5,747,334; 5,756,291; 5,780,225; 5,824,483; 5,840,479; 5,858,670; 5,922,254; 5,922,545; 5,939,598; 5,948,635; 6,057,098; 6,107,059; 6,156,511; 6,168,912; 6,174,708; 6,180,348; 6,214,553; 6,225,447; and 6,593,081.
Wakita et al. (2005) Nature Med 11:791-796.
Wong et al. (2000) Am J Public Health 90:1562-1569.
Yokota et al. (1992) Cancer Res 52:3402-3408.
Zhong et al. (2005) Proc Natl Acad Sci USA 102:9294-9299.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method for predicting a tendency to develop steatosis in a subject infected with hepatitis C virus (HCV), the method comprising:
(a) isolating from the subject a biological sample comprising an HCV Core polypeptide and/or a nucleic acid molecule encoding an HCV Core polypeptide; and
(b) identifying the amino acids in the HCV Core polypeptide and/or encoded by the nucleic acid molecule in the biological sample corresponding to positions 182/186 of SEQ ID NO: 2;
whereby a tendency to develop steatosis in the subject is predicted when the amino acids corresponding to positions 182/186 of SEQ ID NO: 2 in the biological sample are either phenylalanine/valine or leucine/isoleucine.

2. The method of claim 1, wherein the biological sample is selected from the group consisting of a blood sample or a biopsy.

3. The method of claim 1, wherein the biological sample comprises an HCV virion, an HCV genomic RNA molecule, or an RNA molecule encoded by a hepatitis C virus (HCV) genomic RNA molecule.

4. The method of claim 1, wherein the identifying is by nucleic acid sequencing and/or amino acid sequencing.

5. The method of claim 1, wherein the identifying is by contacting the biological sample with an antibody that differentiates between a hepatitis C virus (HCV) Core polypeptide that has a phenylalanine/valine (FV) or a leucine/isoleucine (LI) amino acid pair at positions corresponding to amino acids 182/186 of SEQ ID NO: 2 and an HCV Core polypeptide that does not have an FV or an LI amino acid pair at positions corresponding to amino acids 182/186 of SEQ ID NO: 2.

6. The method of claim 5, wherein the contacting is performed with the hepatitis C virus (HCV) Core polypeptide in solution in the biological sample.

7. The method of claim 5, wherein the contacting is performed subsequent to transferring the hepatitis C virus (HCV) Core polypeptide to a solid support.

8. The method of claim 5, wherein the antibody comprises a detectable label comprising a moiety selected from the group consisting of a light-absorbing dye, a fluorescent dye, a radioactive label, an enzyme, an epitope tag, and biotin.

9. A method for screening for a candidate molecule that modulates lipid accumulation in a cell, the method comprising:
(a) providing a cell infected with hepatitis C virus (HCV), wherein the HCV present therein encodes a Core polypeptide comprising a phenylalanine/valine (FV) or a leucine/isoleucine (LI) amino acid pair at positions corresponding to amino acids 182/186 of SEQ ID NO: 2;
(b) contacting the cell with a candidate molecule under conditions sufficient to allow the candidate molecule to interact with the HCV Core polypeptide and/or to interact with a molecule that interacts with the HCV Core polypeptide in the cell;
(c) quantifying lipid accumulation in the cell; and
(d) comparing lipid accumulation in the cell in the presence of the candidate molecule to lipid accumulation in the cell in the absence of the candidate molecule.

10. The method of claim 9, wherein the candidate molecule is provided in the form of a library.

11. The method of claim 10, wherein the library comprises ten or more diverse molecules.

12. The method of claim 11, wherein the library of diverse molecules comprises a library of one hundred or more diverse molecules.

13. The method of claim 12, wherein the library of diverse molecules comprises a library of a billion or more diverse molecules.

14. The method of claim 1, wherein the library of diverse molecules comprises a library of molecules selected from the group consisting of peptides, peptide mimetics, proteins, antibodies and/or fragments and/or derivatives thereof, small molecules, nucleic acids, and combinations thereof.

15. The method of claim 14, wherein the library of diverse molecules comprises a library of peptides, antibodies and/or fragments and/or derivatives thereof, small molecules, or a combination thereof.

16. A molecule identified by the method claim 9.

17. A method for modulating lipid accumulation in a cell in a subject, the method comprising administering a therapeutically effective amount of a composition comprising the molecule of claim 16.

18. The method of claim 17, wherein the lipid accumulation is associated with steatosis.

19. The method of claim 18, wherein the steatosis comprises lipid accumulation in the liver of the subject.

20. The method of claim 18, wherein the steatosis is incident to infection with HCV.

21. The method of claim 17, wherein the subject is a mammal.

22. The method of claim 21, wherein the mammal is a human.

23. The method of claim 22, wherein the human is infected with hepatitis C virus (HCV).

24. The method of claim 17, wherein the administering is by a route selected from the group consisting of oral, intravenous, intramuscular, transdermal, and inhalation.
